SEQUENCE LISTING (81 sequences)

SEQ ID NO: 1 - protein, 522 aa, Homo sapiens
MNINDLKLTLSKAGQEHLLRFWNELEEAQQVELYAELQAMNFEELNFFFQKAIEGFNQSSHQKNVDARMEPVPREVLGSATRDQDQLQAWESEGLFQISQNKVAVLLLAGGQGTRLGVAYPKGMYDVGLPSRKTLFQIQAERILKLQQVAEKYYGNKCIIPWYIMTSGRTMESTKEFFTKHKYFGLKKENVIFFQQGMLPAMSFDGKIILEEKNKVSMAPDGNGGLYRALAAQNIVEDMEQRGIWSIHVYCVDNILVKVADPRFIGFCIQKGADCGAKVVEKTNPTEPVGVVCRVDGVYQVVEYSEISLATAQKRSSDGRLLFNAGNIANHFFTVPFLRDVVNVYEPQLQHHVAQKKIPYVDTQGQLIKPDKPNGIKMEKFVFDIFQFAKKFVVYEVLREDEFSPLKNADSQNGKDNPTTARHALMSLHHCWVLNAGGHFIDENGSRLPAIPRSATNGKSETITADVNHNLKDANDVPIQCEISPLISYAGEGLESYVADKEFHAPLIIDENGVHELVKNGI

SEQ ID NO: 2 - protein, 23 aa, synthetic construct
ACGAACCCTACAGAACCAGTTGG

SEQ ID NO: 3 - protein, 19 aa, synthetic construct
GAAAAGGTGGACGCACGAA

SEQ ID NO: 4 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 5 - protein, 287 aa, Homo sapiens
GLHNHHPIKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSADVPKPSISSNNSKPVEDKDAVAFHCEPEAQNTTYLWWVNGQSLPVSPRLQLSNGNRTLTLFNVTRNDARAYVCGIQNSVSANRSDPVTLDVLYGPDTPIISPPP

SEQ ID NO: 6 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 7 - protein, 537 aa, Homo sapiens
MSAPKLLSLGCIFFPLLLFQQARAQFPRQCATVEALRSGMCCPDLSPVSGPGTDRCGSSSGRGRCEAVTADSRPHSPQYPHDGRDDREVWPLRFFNRTCHCNGNFSGHNCGTCRPGWRGAACDQRVLIVRRNLLDLSKEEKNHFVRALDMAKRTTHPLFVIATRRSEEILGPDGNTPQFENISIYNYFVWTHYYSVKKTFLGVGQESFGEVDFSHEGPAFLTWHRYHLLRLEKDMQEMLQEPSFSLPYWNFATGKNVCDICTDDLMGSRSNFDSTLISPNSVFSQWRVVCDSLEDYDTLGTLCNSTEDGPIRRNPAGNVARPMVQRLPEPQDVAQCLEVGLFDTPPFYSNSTNSFRNTVEGYSDPTGKYDPAVRSLHNLAHLFLNGTGGQTHLSPNDPIFVLLHTFTDAVFDEWLRRYNADISTFPLENAPIGHNRQYNMVPFWPPVTNTEMFVTAPDNLGYTYEIQWPSREFSVPEIIAIAVVGALLLVALIFGTASYLIRARRSMDEANQPLLTDQYQCYAEEYEKLQNPNQSVV

SEQ ID NO: 8 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 9 - protein, 5 aa, synthetic construct
TYAMN

SEQ ID NO: 10 - protein, 5 aa, synthetic construct
SYAMN

SEQ ID NO: 11 - protein, 19 aa, synthetic construct
RIRSKYNNYATYYADSVKG

SEQ ID NO: 12 - protein, 14 aa, synthetic construct
HGNFGNSYVSWFAY

SEQ ID NO: 13 - protein, 14 aa, synthetic construct
HTTFPSSYVSYYGY

SEQ ID NO: 14 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 15 - protein, 125 aa, synthetic construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSS

SEQ ID NO: 16 - protein, 14 aa, synthetic construct
GSSTGAVTTSNYAN

SEQ ID NO: 17 - protein, 7 aa, synthetic construct
GTNKRAP

SEQ ID NO: 18 - protein, 9 aa, synthetic construct
ALWYSNLWV

SEQ ID NO: 19 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 20 - protein, 453 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 21 - protein, 453 aa, synthetic construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 22 - protein, 216 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 5 aa, synthetic construct
DYFLH

SEQ ID NO: 24 - protein, 17 aa, synthetic construct
WINPDNGNTVYAQKFQG

SEQ ID NO: 25 - protein, 12 aa, synthetic construct
RDYTYEKAALDY

SEQ ID NO: 26 - protein, 121 aa, synthetic construct
QVQLVQSGAEVKKPGASVKVSCKASGFNIKDYFLHWVRQAPGQGLEWMGWINPDNGNTVYAQKFQGRVTMTADTSTSTVYMELSSLRSEDTAVYYCTRRDYTYEKAALDYWGQGTLVTVSS

SEQ ID NO: 27 - protein, 11 aa, synthetic construct
RASGNIYNYLA

SEQ ID NO: 28 - protein, 7 aa, synthetic construct
DAKTLAD

SEQ ID NO: 29 - protein, 9 aa, synthetic construct
QHFWSLPFT

SEQ ID NO: 30 - protein, 107 aa, synthetic construct
DIQMTQSPSSLSASVGDRVTITCRASGNIYNYLAWYQQKPGKVPKLLIYDAKTLADGVPSRFSGSGSGTDFTLTISSLQPEDVATYYCQHFWSLPFTFGQGTKLEIK

SEQ ID NO: 31 - protein, 674 aa, synthetic construct
QVQLVQSGAEVKKPGASVKVSCKASGFNIKDYFLHWVRQAPGQGLEWMGWINPDNGNTVYAQKFQGRVTMTADTSTSTVYMELSSLRSEDTAVYYCTRRDYTYEKAALDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 32 - protein, 449 aa, synthetic construct
QVQLVQSGAEVKKPGASVKVSCKASGFNIKDYFLHWVRQAPGQGLEWMGWINPDNGNTVYAQKFQGRVTMTADTSTSTVYMELSSLRSEDTAVYYCTRRDYTYEKAALDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 33 - protein, 214 aa, synthetic construct
DIQMTQSPSSLSASVGDRVTITCRASGNIYNYLAWYQQKPGKVPKLLIYDAKTLADGVPSRFSGSGSGTDFTLTISSLQPEDVATYYCQHFWSLPFTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 232 aa, synthetic construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 5 aa, synthetic construct
EFGMN

SEQ ID NO: 37 - protein, 17 aa, synthetic construct
WINTKTGEATYVEEFKG

SEQ ID NO: 38 - protein, 12 aa, synthetic construct
WDFAYYVEAMDY

SEQ ID NO: 39 - protein, 11 aa, synthetic construct
KASAAVGTYVA

SEQ ID NO: 40 - protein, 7 aa, synthetic construct
SASYRKR

SEQ ID NO: 41 - protein, 10 aa, synthetic construct
HQYYTYPLFT

SEQ ID NO: 42 - protein, 121 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 43 - protein, 108 aa, synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 44 - protein, 215 aa, synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 451 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - protein, 214 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 47 - protein, 694 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 5 aa, synthetic construct
DTYMH

SEQ ID NO: 49 - protein, 17 aa, synthetic construct
RIDPANGNSKYVPKFQG

SEQ ID NO: 50 - protein, 12 aa, synthetic construct
FGYYVSDYAMAY

SEQ ID NO: 51 - protein, 15 aa, synthetic construct
RAGESVDIFGVGFLH

SEQ ID NO: 52 - protein, 7 aa, synthetic construct
RASNRAT

SEQ ID NO: 53 - protein, 9 aa, synthetic construct
QQTNEDPYT

SEQ ID NO: 54 - protein, 121 aa, synthetic construct
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSS

SEQ ID NO: 55 - protein, 111 aa, synthetic construct
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIK

SEQ ID NO: 56 - protein, 451 aa, synthetic construct
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 57 - protein, 676 aa, synthetic construct
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 218 aa, synthetic construct
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 232 aa, synthetic construct; identical to SEQ ID NO: 34

SEQ ID NO: 60 - protein, 5 aa, synthetic construct
SYAIS

SEQ ID NO: 61 - protein, 17 aa, synthetic construct
GIIPIFGTANYAQKFQG

SEQ ID NO: 62 - protein, 6 aa, synthetic construct
GLLWNY

SEQ ID NO: 63 - protein, 11 aa, synthetic construct
RASQSVSSNLA

SEQ ID NO: 64 - protein, 7 aa, synthetic construct
GASTTAS

SEQ ID NO: 65 - protein, 11 aa, synthetic construct
QQYNNWPPAYT

SEQ ID NO: 66 - protein, 115 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSS

SEQ ID NO: 67 - protein, 109 aa, synthetic construct
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIK

SEQ ID NO: 68 - protein, 216 aa, synthetic construct
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 445 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 70 - protein, 232 aa, synthetic construct; identical to SEQ ID NO: 34

SEQ ID NO: 71 - protein, 670 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 10 aa, synthetic construct
GFTFSDSWIH

SEQ ID NO: 73 - protein, 18 aa, synthetic construct
AWISPYGGSTYYADSVKG

SEQ ID NO: 74 - protein, 9 aa, synthetic construct
RHWPGGFDY

SEQ ID NO: 75 - protein, 11 aa, synthetic construct
RASQDVSTAVA

SEQ ID NO: 76 - protein, 7 aa, synthetic construct
SASFLYS

SEQ ID NO: 77 - protein, 9 aa, synthetic construct
QQYLYHPAT

SEQ ID NO: 78 - protein, 118 aa, synthetic construct [sequence omitted]

SEQ ID NO: 79 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 80 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0102420 A1
(43) Pub. Date: Apr. 16, 2026

(54) CANCER IMMUNOTHERAPY

(71) Applicant: Hoffmann-La Roche Inc., Little Falls, NJ (US)

(72) Inventors: Stephan Gasser, Fislisbach (CH); Katrin Groebke Zbinden, Liestal (CH); Claudio Murgia, Untersiggenthal (CH); Tobias Schmidt, Zurich (CH); Antonio Sorrentino, Basel (CH); Pablo Umaña, Lachen (CH)

(73) Assignee: Hoffmann-La Roche Inc., Little Falls, NJ (US)

(21) Appl. No.: 19/215,007

(22) Filed: May 21, 2025

Related U.S. Application Data
(63) Continuation of application No. PCT/EP2023/083098, filed on Nov. 27, 2023.

(30) Foreign Application Priority Data
Nov. 29, 2022 (EP) 22210060.4

(51) Int. Cl. A61K 31/7028; A61K 39/00; A61K 39/395; A61P 35/00

(52) U.S. Cl. CPC A61K 31/7028; A61K 39/39558; A61P 35/00; A61K 2039/505

(57) ABSTRACT

The present invention relates to combination therapies employing T cell activation and UAP1 inhibitors, and the use of these combination therapies for the treatment of cancer.

### CROSS REFERENCE TO RELATED APPLICATIONS

This application is a continuation of PCT Application No. PCT/EP2023/083098, filed Nov. 27, 2023, which claims priority to European Application No. 22210060.4, filed Nov. 29, 2022, each of which is incorporated by reference in its entirety.

### SEQUENCE LISTING

This application contains a Sequence Listing, which has been submitted electronically in XML format and is hereby incorporated by reference in its entirety. Said XML copy, created on May 20, 2025, is named P37769-US-Sequence-Listing.xml and 82,732 bytes in size.

### FIELD OF THE INVENTION

The present invention relates to combination therapies employing T cell activation and UAP1 inhibitors, and the use of these combination therapies for the treatment of cancer.

### BACKGROUND

Harnessing patients' own immune system against cancer is a promising clinically proven approach to fight malignancies. Immunotherapies such as immune checkpoint inhibitors are routinely used in the clinics for a decade and provide great benefit for patients. However, many patients either do not respond or show tumor relapse after immunotherapy. This lack of efficacy is attributed to the occurrence of innate or acquired resistance mechanisms that create an obstacle to current immunotherapeutics.

Hence, the development of more efficacious cancer immunotherapeutic agents that either improve patients' response rate or overcome resistance mechanisms is needed.

UAP1 is an enzyme involved in the hexosamine biosynthetic pathway (HBP). The HBP integrates glucose and glutamine metabolism to generate UDP-GlucNAc and UDP-GalNAc, also referred to as UDP-HexNAc. These sugar-nucleotides are used in several biological processes such as N- and O-linked protein glycosylation, glycosaminoglycans (GAGs) and glycosphingolipids (GSLs) biosynthesis. Due to increased glucose intake by tumor cells (Warburg effect), the HBP is often more active in malignant cells compared to healthy cells (Akella et al BMC Biol. 2019 Jul. 4; 17(1):52). Additionally, amplification and/or overexpression of the different enzymes involved in this pathway is observed in cancer (Akella et al BMC Biol. 2019 Jul. 4; 17(1):52). The role of the HBP in modulating the immune system in general and T cell functionality more specifically has not been extensively explored so far.

### DESCRIPTION OF THE INVENTION

The present inventors have found that a UDP-N-acetylhexosamine pyrophosphorylase (UAP1) inhibitor may be used to improve the effect of an immunotherapy, in particular wherein the immunotherapy comprises adoptive cell transfer, administration of monoclonal antibodies, administration of cytokines, administration of a cancer vaccine, T cell engaging therapies, administration of PD-1 axis binding antagonists or any combination thereof.

To unbiasedly identify novel potential immunotherapy targets, a CRISPR/Cas9 knockout screen was conducted in tumor cells using naive T cells and Cibisatamab, a CEA-CD3 T cell bispecific antibody (CEA-TCB). The screen revealed genes with immunoregulatory function. UAP1 was one of the strongest hits in the screen and its role in modulating anti cancer immune response was extensively evaluated and validated.

Using an in vitro model of cancer cell killing by human peripheral blood mononuclear cells, the inventors assessed the effects of reduced UAP1 activity on the treatment with three exemplary T-cell bispecific (TCB) antibodies (CEA-TCB, Tyrp1-TCB and EpCAM-TCB), as examples of tumor surface targeting TCBs, and peptide (SIINFEKL) pulsed target cells, as an example of TCR mediated treatment on T cell activation and target cell killing. The results were confirmed in an in vivo cancer model where a synergistic effect of the combination of UAP1 gene knockout and CEA TCB was seen leading to complete tumor regression compared to incomplete tumor growth control in mice treated with CEA-TCB only.

The data in the present Examples show that inhibition of UAP1 can act synergistically with immunotherapy to enhance T cell activation and tumor cell killing, which could be used as a combination in the treatment or prevention of cancer.

Accordingly, in a first aspect, the present invention provides a UDP-N-acetylhexosamine pyrophosphorylase (UAP1) inhibitor for use in the treatment or prevention of cancer in an individual wherein said treatment comprises
(a) the administration of a UAP1 inhibitor to the individual, and
(b) the administration of an immunotherapy to the individual.

In a further aspect, the present invention provides a method for treatment or prevention of cancer in an individual, wherein said method comprises
(a) the administration of a UDP-N-acetylhexosamine pyrophosphorylase (UAP1) inhibitor to the invidual, and
(b) the administration of an immunotherapy to the individual.

In a further aspect, the present invention provides use of an UDP-N-acetylhexosamine pyrophosphorylase (UAP1) inhibitor in the manufacture of a medicament for the treatment of cancer in an individual wherein said treatment comprises
(a) the administration of a UAP1 inhibitor to the invidual, and
(b) the administration of an immunotherapy to the individual.

In a further aspect, the present invention provides an immunotherapy for use in the treatment of a disease in an individual, wherein said treatment comprises
(a) the administration of the immunotherapy to the individual, and
(b) the administration of a UDP-N-acetylhexosamine pyrophosphorylase (UAP1) inhibitor to the invidual.

Terms are used herein as generally used in the art, unless otherwise defined herein.

In some aspects, the immunotherapy comprises adoptive cell transfer, administration of monoclonal antibodies, administration of cytokines, administration of a cancer vaccine, T cell engaging therapies, administration of PD-1 axis binding antagonists or any combination thereof. The term “UAP1 inhibitor” as used herein, refers to compounds which target, decrease or inhibit UAP1 activity and includes but is not limited to a small-molecule inhibitor. The term UAP1 inhibitor as used herein also includes UAP1 degraders and modulators of UAP1 expression resulting in reduced UAP1 activity (as for example compared to UAP1 activity without administration of the UAP1 inhibitor).

Further provided is the UAP1 inhibitor for use in a method as herein described, wherein the UAP1 inhibitor has a molecular weight of 200-900 dalton. Further provided is the UAP1 inhibitor for use in a method as herein described, wherein the UAP1 inhibitor has an IC50 value below 5 μM, 1 μM, below 500 nM, below 200 nM, below 100 nM, below 50 nM, below 25 nM, below 10 nM, below 5 nM, 2 nM or below 1 nM. Further provided is the UAP1 inhibitor for use in a method as herein described, wherein the UAP1 inhibitor comprises at least one heterocycle.

Further provided is the UAP1 inhibitor for use in a method as herein described, wherein the heterocycle comprises at least two heteroatoms. In some aspects, the inhibition of UAP1 (for example by administration of a UAP1 inhibitor) causes an increase of the activity of an immunotherapy.

An exemplary UAP1 inhibitor used as a tool compound in the appended Examples is Ac4Glc2Bz (compound B of WO2016025790):

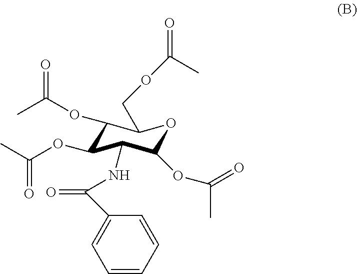

“Activity” of a immunotherapy refers to responses in an individual's body caused by the immunotherapy. Such activity may include cellular response(s) of T cells, particularly CD4+ and/or CD8+ T cells, such as proliferation, differentiation, cytokine secretion, cytotoxic effector molecule release, cytotoxic activity, and expression of activation markers, and/or effects on target cells, particularly target cells (e.g. tumor cells) expressing the target cell antigen of the T cell bispecific antibody, such as lysis of target cells.

In some aspects, (administration of) the UAP1 inhibitor causes increase of the activation of T cells (induced by the immunotherapy).

“Activation of T cells” or “T cell activation” as used herein refers to one or more cellular response of a T lymphocyte, particularly a CD4+ or CD8+ T cell, selected from: proliferation, differentiation, cytokine secretion, cytotoxic effector molecule release, cytotoxic activity, and expression of activation markers. Suitable assays to measure T cell activation are known in the art and described herein. In particular aspects, T cell activation is determined by measuring expression of CD25 and/or CD69 on the T cell, e.g. by flow cytometry.

In some aspects, (administration of) the UAP1 inhibitor causes inhibition of the proliferation of T cells (induced by the immunotherapy). In some aspects, (administration of) the UAP1 inhibitor causes inhibition of the cytotoxic activity of T cells (induced by the immunotherapy).

“Cytotoxic activity” of a T cell refers to the induction of lysis (i.e. killing) of target cells by a T lymphocyte, particularly a CD4+ or CD8+ T cell. Cytotoxic activity typically involves degranulation of the T lymphocyte, associated with the release of cytotoxic effector molecules such as granzyme B and/or perforin from the T lymphocyte.

In some aspects, (administration of) the UAP1 inhibitor causes inhibition of T cell receptor signaling in T cells (induced by the immunotherapy).

By “T cell receptor signaling” is meant activity of the signaling pathway downstream of the T cell receptor (TCR) in a T lymphocyte following engagement of the TCR (such as engagement of the CD3ε subunit of the TCR by a T cell bispecific antibody), involving signaling molecules including tyrosine kinases such as Lek kinase.

In some aspects, (administration of) the UAP1 inhibitor causes increase of cytokine secretion by T cells (induced by the immunotherapy). In some aspects, said cytokine is one or more cytokine selected from the group consisting of IL-2, TNF-α, IFN-γ, IL-6 and IL-1β. In some aspects, said T cells are CD8+ T cells or CD4+ cells.

In some aspects, (administration of) the UAP1 inhibitor causes increase of the level of one or more cytokine in the individual (for example measured in the serum or in a tumor biopsy of the individual). In some aspects, said one or more cytokine is selected from the group consisting of IL-2, TNF-α, and IFN-γ. In some aspects, said increase is sustained after the UAP1 inhibitor has not been administered (to the individual) for a given amount of time. In some aspects, said amount of time is about 1 hour, 2 hours, 3 hours, 4 hours, 5 hours, 6 hours, 7 hours, 8 hours, 12 hours, 16 hours, 20 hours, 24 hours, 36 hours, 48 hours, 72 hours, or 96 hours. In some aspects, said increase is sustained after a subsequent administration of the immunotherapy. Particularly, said increase is sustained even after administration of the UAP1 inhibitor is stopped/no further administration of the UAP1 inhibitor is made. Said increase of the cytokine level is in particular as compared to the level in an individual (including the same individual) without administration of the UAP1 inhibitor (for example the serum level is increased as compared to the serum level without/before administration of the UAP1 inhibitor). Said increase of the cytokine level is in particular as compared to the level in an individual (including the same individual) with administration (in particular first administration) of the immunotherapy but without administration of the UAP1 inhibitor (i.e. in such case the cytokine level is increased as compared to the level with/after administration of the immunotherapy but without/before administration of the UAP1 inhibitor). Cytokine levels can also be measured in a tumor biopsy of an individual to compare a cytokine level in the individual (including the same individual) with administration (in particular first administration) of the immunotherapy but without administration of the UAP1 inhibitor (i.e. in such case the cytokine level is increased as compared to the level with/after administration of the immunotherapy but without/before administration of the UAP1 inhibitor). In some aspects, said increase is clinically meaningful and/or statistically significant.

In some aspects, administration of the UAP1 inhibitor is before the administration of the immunotherapy. In some aspects, administration of the UAP1 inhibitor is concurrent to the administration of the immunotherapy. In some aspects, administration of the UAP1 inhibitor is after the administration of the immunotherapy. Where administration of the UAP1 inhibitor is before or after the administration of the immunotherapy, such administration of the UAP1 inhibitor may be, for example, within about 1 hour, 2 hours, 3 hours, 4 hours, 5 hours, 6 hours, 7 hours, 8 hours, 12 hours, 16 hours, 20 hours or 24 hours before or after, respectively, the administration of the immunotherapy. Administration of the UAP1 inhibitor may be intermittently or continuously. In some aspects, administration of the UAP1 inhibitor is oral.

In some aspects, administration of the UAP1 inhibitor is at a dose sufficient to cause increase of the activity of the immunotherapy. In some aspects, administration of the UAP1 inhibitor is at a dose sufficient to cause increase of the activation of T cells (induced by the immunotherapy). In some aspects, administration of the UAP1 inhibitor is at a dose sufficient to cause increase of the proliferation of T cells (induced by the immunotherapy). In some aspects, administration of the UAP1 inhibitor is at a dose sufficient to cause increase of the cytotoxic activity of T cells (induced by the immunotherapy). In some aspects, administration of the UAP1 inhibitor is at a dose sufficient to cause increase of T cell receptor signaling in T cells (induced by the immunotherapy). In some aspects, administration of the UAP1 inhibitor is at a dose sufficient to cause increase of cytokine secretion by T cells (induced by the immunotherapy). In some aspects, said cytokine is one or more cytokine selected from the group consisting of IL-2, TNF-α, and IFN-γ. In some aspects, said T cells are CD8+ T cells or CD4+ cells. In some aspects, said inhibition is clinically meaningful and/or statistically significant.

Said increase of the cytokine level or cytokine secretion is in particular as compared to the cytokine level or cytokine secretion in an individual (including the same individual) without administration of the UAP1 inhibitor (i.e. in such case the cytokine level is increased as compared to the level without/before administration of the UAP1 inhibitor). Said increase of the cytokine level or cytokine secretion is in particular as compared to the cytokine level or cytokine secretion in an individual (including the same individual) with administration (in particular first administration) of the immunotherapy but without administration of the UAP1 inhibitor (i.e. in such case the cytokine level is increased as compared to the level with/after administration of the immunotherapy but without/before administration of the UAP1 inhibitor). Without said increase, the cytokine level and/or cytokine secretion particularly may be low/decreased in relation to the (administration of) the immunotherapy. In some aspects, said increase is clinically meaningful and/or statistically significant.

In some aspects, administration of the UAP1 inhibitor is at an effective dose.

An “effective amount” or “effective dose” of an agent, e.g. a UAP1 inhibitor or an immunotherapy, refers to an amount effective, at dosages and for periods of time necessary, to achieve the desired therapeutic or prophylactic result. The terms IC50, IC80, IC90 and IC95 as used herein, refer to the inhibitory concentration at which 50%, 80%, 90% and 95% of UAP1 activity is inhibited. In one embodiment, the effective dose is the IC50. In one embodiment, the effective dose is the IC80. In one embodiment, the effective dose is the IC90. In one embodiment, the effective dose is the IC95.

In some embodiments, the UAP1 inhibitor has an IC50 value below 1 μM, below 500 nM, below 200 nM, below 100 nM, below 50 nM, below 25 nM, below 10 nM, below 5 nM, 2 nM or below 1 nM. In some embodiments, the UAP1 inhibitor has an IC80 value below 1 μM, below 500 nM, below 200 nM, below 100 nM, below 50 nM, below 25 nM, below 10 nM, below 5 nM, 2 nM or below 1 nM. In some embodiments, the UAP1 inhibitor has an IC90 value below 1 μM, below 500 nM, below 200 nM, below 100 nM, below 50 nM, below 25 nM, below 10 nM, below 5 nM, 2 nM or below 1 nM. In some embodiments, the UAP1 inhibitor has an IC95 value below 1 μM, below 500 nM, below 200 nM, below 100 nM, below 50 nM, below 25 nM, below 10 nM, below 5 nM, 2 nM or below 1 nM. In some embodiments, the UAP1 inhibitor decreases UAP1 activity at least about 10%, at least about 20%, at least about 30%, at least about 40%, at least about 50%, at least about 60%, at least about 70%, at least about 75%, at least about 80%, at least about 85%, at least about 90%, at least about 95% or at least about 99%. IC50 values can be measured according to the procedure and methods well known in the art.

In some aspects, administration of the UAP1 inhibitor is at a dose of about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 300 mg, 400 mg, 500 mg, 600 mg, 700 mg, 800 mg, 900 mg, 1000 mg or above.

In some aspects, administration of the UAP1 inhibitor is at a dose of between about 1 mg and about 10 g, between about 10 mg and about 5000 mg, between about 50 mg and about 2000 mg or between about 100 mg and about 1000 mg.

In some aspects, administration of the UAP1 inhibitor is daily. In some aspects, administration of the UAP1 inhibitor is once daily. In some aspects, administration of the UAP1 inhibitor is once, twice, three times, four times, five times, six times, seven times, eight times, nine times or ten times, particularly once, twice, three times, four times, five times, six times, seven times, eight times, nine times or ten times in the course of the treatment of the individual with the immunotherapy. In some aspects, administration of the UAP1 inhibitor is for 1 day, 2 days, 3 days, 4 days, 5 days, 6 days, 7 days, 8 days, 9 days or 10 days. In some aspects, administration of the UAP1 inhibitor is once daily for 1 day, 2 days, 3 days, 4 days, 5 days, 6 days, 7 days, 8 days, 9 days or 10 days. In some aspects, administration of the UAP1 inhibitor is associated with the first administration of the immunotherapy. Said first administration is particularly the first administration of the immunotherapy in the course of the treatment of the individual with the immunotherapy. In some aspects, administration of the UAP1 inhibitor is concurrent with the first administration of the immunotherapy. In some aspects, administration of the UAP1 inhibitor is prior to the first administration of the immunotherapy. In some aspects, administration of the UAP1 inhibitor is subsequent to the first administration of the immunotherapy. In some aspects, administration of the UAP1 inhibitor is subsequent to the first administration of the immunotherapy and prior to a second administration of the immunotherapy. Where administration of the UAP1 inhibitor is prior or subsequent to the (first) administration of the immunotherapy, such administration of the UAP1 inhibitor may be, for example, within about 1 hour, 2 hours, 3 hours, 4 hours, 5 hours, 6 hours, 7 hours, 8 hours, 12 hours, 16 hours, 20 hours or 24 hours before or after, respectively, the administration of the immunotherapy.

In some aspects, the administration of the immunotherapy is for a longer period of time than the administration of the UAP1 inhibitor. In some aspects, the administration of the immunotherapy continues after the administration of the UAP1 inhibitor is stopped. In some aspects, the administration of the immunotherapy is a single administration or a repeated administration. In the course of the treatment of the individual with the immunotherapy, the immunotherapy may be administered once or several times. For example, treatment of the individual with the immunotherapy may comprise multiple treatment cycles which each comprise one or more administrations of the immunotherapy. In some aspects, the administration of the immunotherapy comprises a first and a second administration.

For use in the present invention, the immunotherapy would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In some aspects, the administration of the immunotherapy is at an effective dose. For systemic administration, an effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. Dosage amount and interval may be adjusted individually to provide plasma levels of the immunotherapy which are sufficient to maintain therapeutic effect. For example for T cell bispecific antibodies, usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

An effective amount of the immunotherapy may be administered for prevention or treatment of disease. The appropriate route of administration and dosage of the immunotherapy may be determined based on the type of disease to be treated, the type of the immunotherapy, the severity and course of the disease, the clinical condition of the individual, the individual's clinical history and response to the treatment, and the discretion of the attending physician. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The immunotherapy and the UAP1 inhibitor can be administered by any suitable route, and may be administered by the same route of administration or by different routes of administration. In some aspects, the administration of the immunotherapy is parenteral, particularly intravenous.

In some aspects, the administration of the immunotherapy is the first administration of the immunotherapy to the individual, particularly the first administration of the immunotherapy in the course of the treatment of the individual with the immunotherapy.

In some aspects, (administration of) the immunotherapy induces (i.e. causes or increases) the activation of T cells. In some aspects, (administration of) the immunotherapy induces the proliferation of T cells. In some aspects, (administration of) the immunotherapy induces cytotoxic activity of T cells. In some aspects, (administration of) the immunotherapy induces T cell receptor signaling in T cells. In some aspects, (administration of) the immunotherapy induces cytokine secretion by T cells. In some aspects, the cytokine is one or more cytokine selected from the group consisting of IL-2, TNF-α, and IFN-γ. In some aspects, said T cells are CD8+ T cells or CD4+ cells.

In some aspects, administration of the immunotherapy results in activation of T cells, particularly cytotoxic T cells, particularly at the site of the cancer (e.g. within a solid tumor cancer). Said activation may comprise proliferation of T cells, differentiation of T cells, cytokine secretion by T cells, cytotoxic effector molecule release from T cells, cytotoxic activity of T cells, and expression of activation markers by T cells. In some aspects, the administration of the immunotherapy results in an increase of T cell, particularly cytotoxic T cell, numbers at the site of the cancer (e.g. within a solid tumor cancer).

In some aspects, the immunotherapy comprises adoptive cell transfer, administration of monoclonal antibodies, administration of cytokines, administration of a cancer vaccine, T cell engaging therapies, administration of PD-1 axis binding antagonists or any combination thereof.

In the following, T cell engaging therapies that may be used as an immunotherapy in the present invention are further described.

In one aspect, the T cell engaging therapy is a T cell bispecific antibody as further described below. In one aspect, the immunotherapy is a T cell bispecific antibody as further described below.

By “T cell bispecific antibody” abbreviated “TCB” is meant an antibody that is able to bind, including simultaneously bind, to a T cell (typically via an antigenic determinant expressed on the T cell, such as CD3) and to a target cell (typically via an antigenic determinant expressed on the target cell, such as CEA, TYRP1, or EpCAM).

In preferred aspects according to the invention, the T cell bispecific antibody is capable of simultaneous binding to the antigenic determinant on the T cell (i.e. a first antigen such as CD3) and the antigenic determinant on the target cell (i.e. a second antigen such as CEA, TYRP1, or EpCAM). In some aspects, the T cell bispecific antibody is capable of crosslinking the T cell and the target cell by simultaneous binding to CD3 and a target cell antigen. In even more preferred aspects, such simultaneous binding results in lysis of the target cell, particularly a target cell antigen (e.g. CEA, TYRP1, or EpCAM)-expressing tumor cell. In some aspects, such simultaneous binding results in activation of the T cell. In some aspects, such simultaneous binding results in a cellular response of the T cell, selected from the group of: proliferation, differentiation, cytokine secretion, cytotoxic effector molecule release, cytotoxic activity, and expression of activation markers. In some aspects, binding of the T cell bispecific antibody to CD3 without simultaneous binding to the target cell antigen does not result in T cell activation. In some aspects, the T cell bispecific antibody is capable of re-directing cytotoxic activity of a T cell to a target cell. In preferred aspects, said re-direction is independent of MHC-mediated peptide antigen presentation by the target cell and and/or specificity of the T cell.

The term “bispecific” means that the antibody is able to bind to at least two distinct antigenic determinants. Typically, a bispecific antibody comprises two antigen binding sites, each of which is specific for a different antigenic determinant. In certain aspects, the bispecific antibody is capable of simultaneously binding two antigenic determinants, particularly two antigenic determinants expressed on two distinct cells.

As used herein, the term “antigenic determinant” is synonymous with “antigen” and “epitope”, and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

As used herein, the term “antigen binding moiety” refers to a polypeptide molecule that binds, including specifically binds, to an antigenic determinant. In some aspects, an antigen binding moiety is able to direct the entity to which it is attached (e.g. a second antigen binding moiety) to a target site, for example to a specific type of tumor cell bearing the antigenic determinant. In further aspects, an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain aspects, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

By “specific binding” is meant that the binding is selective for the antigen and can be discriminated from unwanted or non-specific interactions. The term “bind” or “binding” herein generally refers to “specific binding”. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In some aspects, the extent of binding of an antigen binding moiety to an unrelated protein is less than about 10% of the binding of the antigen binding moiety to the antigen as measured, e.g., by SPR. In certain aspects, an antigen binding moiety that binds to the antigen, or an antibody comprising that antigen binding moiety, has a dissociation constant (K_{D}) of ≤1 μM, <100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In some aspects, CD3 is human CD3, particularly the epsilon subunit of human CD3 (CD3F). The amino acid sequence of human CD3ε is shown in UniProt (www.uniprot.org) accession no. P07766 (version 144), or NCBI (www.ncbi.nlm.nih.gov/) RefSeq NP_000724.1. See also SEQ ID NO: 4.

A “target cell antigen” as used herein refers to an antigenic determinant presented on the surface of a target cell, for example a cell in a tumor such as a cancer cell or a cell of the tumor stroma (in that case a “tumor cell antigen”). Preferably, the target cell antigen is not CD3, and/or is expressed on a different cell than CD3. In some aspects, the target cell antigen is CEA, particularly human CEA. In other aspects, the target cell antigen is TYRP1, particularly human TYRPL. In some aspects, the target cell antigen is EpCAM, particularly human EpCAM.

As used herein, the terms “first”, “second” or “third” with respect to antigen binding moieties etc., are used for convenience of distinguishing when there is more than one of each type of moiety. Use of these terms is not intended to confer a specific order or orientation of the bispecific antibody unless explicitly so stated.

The term “valent” as used herein denotes the presence of a specified number of antigen binding sites in an antibody. As such, the term “monovalent binding to an antigen” denotes the presence of one (and not more than one) antigen binding site specific for the antigen in the antibody.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The terms “full length antibody”, “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Plickthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991). As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence and which determine antigen binding specificity, for example “complementarity determining regions” (“CDRs”). Generally, antibodies comprise six CDRs; three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1(L1)-FR2-H2(L2)-FR3-H3(L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “Fab molecule” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some aspects the modification promoting association comprises an amino acid mutation in the Fc domain, specifically an amino acid substitution. In particular aspects, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36, and is publicly available from http://fasta.bioch.virginia.edu/fasta_www2/fasta_down.shtml. Alternatively, a public server accessible at http://fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), Fc≢RIIa (CD32), and FcαRI (CD89).

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

By “fused” is meant that the components (e.g. a Fab molecule and an Fc domain subunit) are linked by peptide bonds, either directly or via one or more peptide linkers.

In particular aspects, the T cell bispecific antibody binds to CD3 and a target cell antigen.

Accordingly, in some aspects, the T cell bispecific antibody comprises an antigen binding moiety that binds to CD3 and an antigen binding moiety that binds to a target cell antigen.

In some aspects, the first and/or the second antigen binding moiety is a Fab molecule. In some aspects, the first antigen binding moiety is a crossover Fab molecule wherein either the variable or the constant regions of the Fab light chain and the Fab heavy chain are exchanged. In such aspects, the second antigen binding moiety preferably is a conventional Fab molecule.

In some aspects wherein the first and the second antigen binding moiety of the T cell bispecific antibody are both Fab molecules, and in one of the antigen binding moieties (particularly the first antigen binding moiety) the variable domains VL and VH of the Fab light chain and the Fab heavy chain are replaced by each other,
i) in the constant domain CL of the first antigen binding moiety the amino acid at position 124 is substituted by a positively charged amino acid (numbering according to Kabat), and wherein in the constant domain CH1 of the first antigen binding moiety the amino acid at position 147 or the amino acid at position 213 is substituted by a negatively charged amino acid (numbering according to Kabat EU index); or
ii) in the constant domain CL of the second antigen binding moiety the amino acid at position 124 is substituted by a positively charged amino acid (numbering according to Kabat), and wherein in the constant domain CH1 of the second antigen binding moiety the amino acid at position 147 or the amino acid at position 213 is substituted by a negatively charged amino acid (numbering according to Kabat EU index).

The T cell bispecific antibody does not comprise both modifications mentioned under i) and ii). The constant domains CL and CH1 of the antigen binding moiety having the VH/VL exchange are not replaced by each other (i.e. remain unexchanged).

In more specific aspects,
i) in the constant domain CL of the first antigen binding moiety the amino acid at position 124 is substituted independently by lysine (K), arginine (R) or histidine (H) (numbering according to Kabat), and in the constant domain CH1 of the first antigen binding moiety the amino acid at position 147 or the amino acid at position 213 is substituted independently by glutamic acid (E), or aspartic acid (D) (numbering according to Kabat EU index); or
ii) in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 or the amino acid at position 213 is substituted independently by glutamic acid (E), or aspartic acid (D) (numbering according to Kabat EU index).

In some aspects, in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 or the amino acid at position 213 is substituted independently by glutamic acid (E), or aspartic acid (D) (numbering according to Kabat EU index).

In further aspects, in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 is substituted independently by glutamic acid (E), or aspartic acid (D) (numbering according to Kabat EU index).

In preferred aspects, in the constant domain CL of the second antigen binding moiety the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second antigen binding moiety the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In some aspects, in the constant domain CL of the second antigen binding moiety the amino acid at position 124 is substituted by lysine (K) (numbering according to Kabat) and the amino acid at position 123 is substituted by lysine (K) (numbering according to Kabat), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In some aspects, in the constant domain CL of the second antigen binding moiety the amino acid at position 124 is substituted by lysine (K) (numbering according to Kabat) and the amino acid at position 123 is substituted by arginine (R) (numbering according to Kabat), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In particular aspects, if amino acid substitutions according to the above aspects are made in the constant domain CL and the constant domain CH1 of the second antigen binding moiety, the constant domain CL of the second antigen binding moiety is of kappa isotype.

In some aspects, the first and the second antigen binding moiety are fused to each other, optionally via a peptide linker.

In some aspects, the first and the second antigen binding moiety are each a Fab molecule and either (i) the second antigen binding moiety is fused at the C-terminus of the Fab heavy chain to the N-terminus of the Fab heavy chain of the first antigen binding moiety, or (ii) the first antigen binding moiety is fused at the C-terminus of the Fab heavy chain to the N-terminus of the Fab heavy chain of the second antigen binding moiety.

In some aspects, the T cell bispecific antibody provides monovalent binding to CD3.

In particular aspects, the T cell bispecific antibody comprises a single antigen binding moiety that binds to CD3, and two antigen binding moieties that bind to the target cell antigen. Thus, in some aspects, the T cell bispecific antibody comprises a third antigen binding moiety, particularly a Fab molecule, more particularly a conventional Fab molecule, that binds to the target antigen. The third antigen binding moiety may incorporate, singly or in combination, all of the features described herein in relation to the second antigen binding moiety (e.g. the CDR sequences, variable region sequences, and/or amino acid substitutions in the constant regions). In some aspects, the third antigen moiety is identical to the first antigen binding moiety (e.g. is also a conventional Fab molecule and comprises the same amino acid sequences).

In particular aspects, the T cell bispecific antibody further comprises an Fc domain composed of a first and a second subunit. In some aspects, the Fc domain is an IgG Fc domain. In particular aspects, the Fc domain is an IgG₁ Fc domain. In other aspects, the Fc domain is an IgG₄ Fc domain. In more specific aspects, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In further particular aspects, the Fc domain is a human Fc domain. In particularly preferred aspects, the Fc domain is a human IgG₁ Fc domain. An exemplary sequence of a human IgG₁ Fc region is given in SEQ ID NO: 27.

In some aspects wherein the first, the second and, where present, the third antigen binding moiety are each a Fab molecule, (a) either (i) the second antigen binding moiety is fused at the C-terminus of the Fab heavy chain to the N-terminus of the Fab heavy chain of the first antigen binding moiety and the first antigen binding moiety is fused at the C-terminus of the Fab heavy chain to the N-terminus of the first subunit of the Fc domain, or (ii) the first antigen binding moiety is fused at the C-terminus of the Fab heavy chain to the N-terminus of the Fab heavy chain of the second antigen binding moiety and the second antigen binding moiety is fused at the C-terminus of the Fab heavy chain to the N-terminus of the first subunit of the Fc domain; and (b) the third antigen binding moiety, where present, is fused at the C-terminus of the Fab heavy chain to the N-terminus of the second subunit of the Fc domain.

In some aspects, the T cell bispecific antibody essentially consists of the first, the second and the third antigen binding moiety (particularly Fab molecule), the Fc domain composed of a first and a second subunit, and optionally one or more peptide linkers.

The components of the T cell bispecific antibody may be fused to each other directly or, preferably, via one or more suitable peptide linkers. Where fusion of a Fab molecule is to the N-terminus of a subunit of the Fc domain, it is typically via an immunoglobulin hinge region.

The antigen binding moieties may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ, G₄(SG₄)ₙ or (G₄S)ₙG₅ peptide linkers. “n” is generally an integer from 1 to 10, typically from 2 to 4. In some aspects, said peptide linker has a length of at least 5 amino acids, in some aspects a length of 5 to 100, in further aspects of 10 to 50 amino acids. In some aspects said peptide linker is (GxS)ₙ or (GXS)ₙGₘ with G=glycine, S=serine, and (x=3, n=3, 4, 5 or 6, and m=0, 1, 2 or 3) or (x=4, n=1, 2, 3, 4 or 5 and m=0, 1, 2, 3, 4 or 5), in some aspects x=4 and n=2 or 3, in further aspects x=4 and n=2, in yet further aspects x=4, n=1 and m=5. In some aspects, said peptide linker is (G₄S)₂. In other aspects, said peptide linker is G₄SG₅. Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In particular aspects, the Fc domain comprises a modification promoting the association of the first and the second subunit of the Fc domain. The site of most extensive protein-protein interaction between the two subunits of a human IgG Fc domain is in the CH3 domain. Thus, in some aspects, said modification is in the CH3 domain of the Fc domain.

In specific aspects, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in some aspects, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g. by site-specific mutagenesis, or by peptide synthesis.

In specific such aspects, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In further aspects, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numbering according to Kabat EU index). In preferred aspects, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some aspects, the Fc domain comprises one or more amino acid substitution that reduces binding to an Fc receptor and/or effector function.

In particular aspects, the Fc receptor is an Fcγ receptor. In some aspects, the Fc receptor is a human Fc receptor. In some aspects, the Fc receptor is an activating Fc receptor. In specific aspects, the Fc receptor is an activating human Fcγ receptor, more specifically human FcγRIIIa, FcγRI or FcγRIIa, most specifically human FcγRIIIa. In some aspects, the effector function is one or more selected from the group of complement dependent cytotoxicity (CDC), antibody-dependent cell-mediated cytotoxicity (ADCC), antibody-dependent cellular phagocytosis (ADCP), and cytokine secretion. In particular aspects, the effector function is ADCC.

Typically, the same one or more amino acid substitution is present in each of the two subunits of the Fc domain. In some aspects, the one or more amino acid substitution reduces the binding affinity of the Fc domain to an Fc receptor. In some aspects, the one or more amino acid substitution reduces the binding affinity of the Fc domain to an Fc receptor by at least 2-fold, at least 5-fold, or at least 10-fold.

In some aspects, the Fc domain comprises an amino acid substitution at a position selected from the group of E233, L234, L235, N297, P331 and P329 (numberings according to Kabat EU index). In more specific aspects, the Fc domain comprises an amino acid substitution at a position selected from the group of L234, L235 and P329 (numberings according to Kabat EU index). In some aspects, the Fc domain comprises the amino acid substitutions L234A and L235A (numberings according to Kabat EU index). In some such aspects, the Fc domain is an IgG₁ Fc domain, particularly a human IgG₁ Fc domain. In some aspects, the Fc domain comprises an amino acid substitution at position P329. In more specific aspects, the amino acid substitution is P329A or P329G, particularly P329G (numberings according to Kabat EU index). In some aspects, the Fc domain comprises an amino acid substitution at position P329 and a further amino acid substitution at a position selected from E233, L234, L235, N297 and P331 (numberings according to Kabat EU index). In more specific aspects, the further amino acid substitution is E233P, L234A, L235A, L235E, N297A, N297D or P331S. In particular aspects, the Fc domain comprises amino acid substitutions at positions P329, L234 and L235 (numberings according to Kabat EU index). In more particular aspects, the Fc domain comprises the amino acid mutations L234A, L235A and P329G (“P329G LALA”, “PGLALA” or “LALAPG”). Specifically, in preferred aspects, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In some such aspects, the Fc domain is an IgG₁ Fc domain, particularly a human IgG₁ Fc domain.

In some aspects, the target cell antigen of the T cell bispecific antibody is carcinoembryonic antigen (CEA).

“Carcinoembryonic antigen” or “CEA” (also known as Carcinoembryonic antigen-related cell adhesion molecule 5 (CEACAM5)) refers to any native CEA from any vertebrate source, including mammals such as primates (e.g. humans), non-human primates (e.g. cynomolgus monkeys) and rodents (e.g. mice and rats), unless otherwise indicated. The term encompasses “full-length,” unprocessed CEA as well as any form of CEA that results from processing in the cell. The term also encompasses naturally occurring variants of CEA, e.g., splice variants or allelic variants. In some aspects, CEA is human CEA. The amino acid sequence of human CEA is shown in UniProt (www.uniprot.org) accession no. P06731, or NCBI (www.ncbi.nlm.nih.gov/) RefSeq NP_004354.2. See also SEQ ID NO: 5. In some aspects, CEA is cell membrane-bound CEA. In some aspects, CEA is CEA expressed on the surface of a cell, e.g. a cancer cell.

Useful T cell bispecific antibodies for the present invention that bind to CEA are described e.g. in PCT publication no. WO 2014/131712 (incorporated herein by reference in its entirety).

Is some aspects, the T cell bispecific antibody comprises a first antigen binding moiety that binds to CD3, and a second antigen binding moiety that binds to CEA.

In some aspects, the first antigen binding moiety comprises a heavy chain variable region comprising the heavy chain CDR (HCDR) 1 of SEQ ID NO: 9, the HCDR2 of SEQ ID NO: 11, and the HCDR3 of SEQ ID NO: 12; and a light chain variable region comprising the light chain CDR (LCDR) 1 of SEQ ID NO: 16, the LCDR2 of SEQ ID NO: 17 and the LCDR3 of SEQ ID NO: 18.

In some aspects, the second antigen binding moiety comprises a heavy chain variable region comprising the heavy chain CDR (HCDR) 1 of SEQ ID NO: 36, the HCDR2 of SEQ ID NO: 37, and the HCDR3 of SEQ ID NO: 38; and a light chain variable region comprising the light chain CDR (LCDR) 1 of SEQ ID NO: 39, the LCDR2 of SEQ ID NO: 40 and the LCDR3 of SEQ ID NO: 41.

In some aspects, the CEA CD3 bispecific antibody comprises
(i) a first antigen binding moiety that binds to CD3 and comprises a heavy chain variable region comprising the heavy chain CDR (HCDR) 1 of SEQ ID NO: 9, the HCDR2 of SEQ ID NO: 11, and the HCDR3 of SEQ ID NO: 12; and a light chain variable region comprising the light chain CDR (LCDR) 1 of SEQ ID NO: 16, the LCDR2 of SEQ ID NO: 17 and the LCDR3 of SEQ ID NO: 18; and
(ii) a second antigen binding moiety that binds to CEA and comprises a heavy chain variable region comprising the heavy chain CDR (HCDR) 1 of SEQ ID NO: 36, the HCDR2 of SEQ ID NO: 37, and the HCDR3 of SEQ ID NO: 38; and a light chain variable region comprising the light chain CDR (LCDR) 1 of SEQ ID NO: 39, the LCDR2 of SEQ ID NO: 40 and the LCDR3 of SEQ ID NO: 41.

In some aspects, the first antigen binding moiety comprises a heavy chain variable region sequence that is at least about 95%, 96%, 97%, 98%, 99% or 100% identical to the amino acid sequence of SEQ ID NO: 14 and a light chain variable region sequence that is at least about 95%, 96%, 97%, 98%, 99% or 100% identical to the amino acid sequence of SEQ ID NO: 19. In some aspects, the first antigen binding moiety comprises the heavy chain variable region sequence of SEQ ID NO: 14 and the light chain variable region sequence of SEQ ID NO: 19.

In some aspects, the second antigen binding moiety comprises a heavy chain variable region sequence that is at least about 95%, 96%, 97%, 98%, 99% or 100% identical to the amino acid sequence of SEQ ID NO: 42 and a light chain variable region sequence that is at least about 95%, 96%, 97%, 98%, 99% or 100% identical to the amino acid sequence of SEQ ID NO: 43. In some aspects, the second antigen binding moiety comprises the heavy chain variable region sequence of SEQ ID NO: 42 and the light chain variable region sequence of SEQ ID NO: 43.

In some aspects, the T cell bispecific antibody comprises a third antigen binding moiety that binds to CEA and/or an Fc domain composed of a first and a second subunit, as described herein.

In preferred aspects, the T cell bispecific antibody comprises
(i) a first antigen binding moiety that binds to CD3, comprising a heavy chain variable region comprising the heavy chain CDR (HCDR) 1 of SEQ ID NO: 9, the HCDR2 of SEQ ID NO: 11, and the HCDR3 of SEQ ID NO: 12; and a light chain variable region comprising the light chain CDR (LCDR) 1 of SEQ ID NO: 16, the LCDR2 of SEQ ID NO: 17 and the LCDR3 of SEQ ID NO: 18, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety that bind to CEA, comprising a heavy chain variable region comprising the heavy chain CDR (HCDR) 1 of SEQ ID NO: 36, the HCDR2 of SEQ ID NO: 37, and the HCDR3 of SEQ ID NO: 38; and a light chain variable region comprising the light chain CDR (LCDR) 1 of SEQ ID NO: 39, the LCDR2 of SEQ ID NO: 40 and the LCDR3 of SEQ ID NO: 41, wherein the second and third antigen binding moiety are each a Fab molecule, particularly a conventional Fab molecule;
(iii) an Fc domain composed of a first and a second subunit, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In some aspects, the first antigen binding moiety of the T cell bispecific antibody (that binds to CEA and CD3) comprises a heavy chain variable region sequence that is at least about 95%, 96%, 97%, 98%, 99% or 100% identical to the amino acid sequence of SEQ ID NO: 14 and a light chain variable region sequence that is at least about 95%, 96%, 97%, 98%, 99% or 100% identical to the amino acid sequence of SEQ ID NO: 19. In some aspects, the first antigen binding moiety comprises the heavy chain variable region sequence of SEQ ID NO: 14 and the light chain variable region sequence of SEQ ID NO: 19.

In some aspects, the second and (where present) third antigen binding moiety of the T cell bispecific antibody (that binds to CEA and CD3) comprise a heavy chain variable region sequence that is at least about 95%, 96%, 97%, 98%, 99% or 100% identical to the amino acid sequence of SEQ ID NO: 42 and a light chain variable region sequence that is at least about 95%, 96%, 97%, 98%, 99% or 100% identical to the amino acid sequence of SEQ ID NO: 43. In some aspects, the second and (where present) third antigen binding moiety comprise the heavy chain variable region of SEQ ID NO: 42 and the light chain variable region of SEQ ID NO: 43.

The Fc domain according to the above aspects may incorporate, singly or in combination, all of the features described hereinabove in relation to Fc domains.

In some aspects, the Fc domain of the T cell bispecific antibody (that binds to CEA and CD3) comprises a modification promoting the association of the first and the second subunit of the Fc domain, and/or the Fc domain comprises one or more amino acid substitution that reduces binding to an Fc receptor and/or effector function.

In some aspects, the antigen binding moieties and the Fc region are fused to each other by peptide linkers, in particular the peptide linkers as described above.

In some aspects, the T cell bispecific antibody (that binds to CEA and CD3) comprises a polypeptide (particularly two polypeptides) comprising a sequence that is at least 80%, 85%, 90%, 95%, 96%, 97%, 98%, or 99% identical to the sequence of SEQ ID NO: 44, a polypeptide comprising a sequence that is at least 80%, 85%, 90%, 95%, 96%, 97%, 98%, or 99% identical to the sequence of SEQ ID NO: 45, a polypeptide comprising a sequence that is at least 80%, 85%, 90%, 95%, 96%, 97%, 98%, or 99% identical to the sequence of SEQ ID NO: 46, and a polypeptide comprising a sequence that is at least 80%, 85%, 90%, 95%, 96%, 97%, 98%, or 99% identical to the sequence of SEQ ID NO: 47. In some aspects, the T cell bispecific antibody (that binds to CEA and CD3) comprises a polypeptide (particularly two polypeptides) comprising the sequence of SEQ ID NO: 44, a polypeptide comprising the sequence of SEQ ID NO: 45, a polypeptide comprising the sequence of SEQ ID NO: 46, and a polypeptide comprising the sequence of SEQ ID NO: 47.

In preferred aspects, the T cell bispecific antibody is cibisatamab (WHO Drug Information (International Nonproprietary Names for Pharmaceutical Substances), Recommended INN: List 80, 2018, vol. 32, no. 3, p. 438).

The T cell bispecific antibody cibisatamab (RG7802, R06958688, CEA-TCB) is a novel T-cell activating bispecific antibody targeting carcinoembryonic antigen (CEA) on tumor cells and CD3 on T-cells, that redirects T cells independently of their T cell receptor specificity to tumor cells expressing the CEA glycoprotein at the cell surface (Bacac et al., Oncoimmunology. 2016; 5(8):1-30). A major advantage of T cell redirecting bispecific antibodies is that they mediate cancer cell recognition by T cells independently of neoantigen load. CEA is overexpressed on the cell surface of many colorectal cancers (CRC) and cibisatamab is hence a promising immunotherapy agent for non-hypermutated microsatellite stable (MSS) CRCs.

Cibisatamab has a single binding site for the CD3 epsilon chain on T cells and two CEA binding sites which tune the binding avidity to cancer cells with moderate to high CEA cell surface expression (Bacac et al., Clin Cancer Res. 2016; 22(13):3286-97). This avoids targeting of healthy epithelial cells with low CEA expression levels, which are physiologically present in some tissues. Binding of cibisatamab to CEA on the surface of cancer cells and of CD3 on T cells triggers T cell activation, cytokine secretion and cytotoxic granule release. The phase I trial of cibisatamab in patients with CEA expressing metastatic CRCs that had failed at least two prior chemotherapy regimens showed antitumor activity with radiological shrinkage in 11% ( 4/36) and 50% ( 5/10) of patients treated with monotherapy or in combination with PD-L1-inhibiting antibodies, respectively (Argilds et al., Ann Oncol. 2017 Jun. 1; 28(suppl_3):mdx302.003-mdx302.003; Tabernero et al., J Clin Oncol. 2017 May 20;35(15_suppl):3002). Based on these results, CEA is one of the most promising target antigens for immunotherapy in MSS CRCs. Although some patients in this dose escalation trial were treated with a dose below the final recommended dose, the response rates nevertheless indicate that a subgroup of tumors is resistant to treatment.

In some aspects, the target cell antigen of the T cell bispecific antibody is EpCAM.

“EpCAM”, also known as “epitherlial cellular adhesion molecule”, refers to any native EpCAM from any vertebrate source, including mammals such as primates (e.g. humans), non-human primates (e.g. cynomolgus monkeys) and rodents (e.g. mice and rats), unless otherwise indicated. The term encompasses “full-length,” unprocessed EpCAM as well as any form of EpCAM that results from processing in the cell. The term also encompasses naturally occurring variants of EpCAM, e.g., splice variants or allelic variants. In some aspects, EpCAM is human EpCAM. Human EpCAM is described in UniProt (www.uniprot.org) accession no. P16422 (entry version 207), and an amino acid sequence of human EpCAM is also shown in SEQ ID NO: 6.

In some aspects, the T cell bispecific antibody comprises a first antigen binding moiety that binds to CD3, and a second antigen binding moiety that binds to EpCAM.

In some aspects, the first antigen binding moiety comprises a heavy chain variable region comprising the heavy chain CDR (HCDR) 1 of SEQ ID NO: 9, the HCDR2 of SEQ ID NO: 11, and the HCDR3 of SEQ ID NO: 12; and a light chain variable region comprising the light chain CDR (LCDR) 1 of SEQ ID NO: 16, the LCDR2 of SEQ ID NO: 17 and the LCDR3 of SEQ ID NO: 18.

In some aspects, the second antigen binding moiety comprises a heavy chain variable region comprising the heavy chain CDR (HCDR) 1 of SEQ ID NO: 60, the HCDR2 of SEQ ID NO: 61, and the HCDR3 of SEQ ID NO: 62; and a light chain variable region comprising the light chain CDR (LCDR) 1 of SEQ ID NO: 63, the LCDR2 of SEQ ID NO: 64 and the LCDR3 of SEQ ID NO: 65.

In some aspects, the T cell bispecific antibody comprises
(i) a first antigen binding moiety that binds to CD3 and comprises a heavy chain variable region comprising the heavy chain CDR (HCDR) 1 of SEQ ID NO: 9, the HCDR2 of SEQ ID NO: 11, and the HCDR3 of SEQ ID NO: 12; and a light chain variable region comprising the light chain CDR (LCDR) 1 of SEQ ID NO: 16, the LCDR2 of SEQ ID NO: 17 and the LCDR3 of SEQ ID NO: 18; and
(ii) a second antigen binding moiety that binds to EpCAM and comprises a heavy chain variable region comprising the heavy chain CDR (HCDR) 1 of SEQ ID NO: 60, the HCDR2 of SEQ ID NO: 61, and the HCDR3 of SEQ ID NO: 62; and a light chain variable region comprising the light chain CDR (LCDR) 1 of SEQ ID NO: 63, the LCDR2 of SEQ ID NO: 64 and the LCDR3 of SEQ ID NO: 65.

In some aspects, the first antigen binding moiety comprises a heavy chain variable region sequence that is at least about 95%, 96%, 97%, 98%, 99% or 100% identical to the amino acid sequence of SEQ ID NO: 14 and a light chain variable region sequence that is at least about 95%, 96%, 97%, 98%, 99% or 100% identical to the amino acid sequence of SEQ ID NO: 19. In some aspects, the first antigen binding moiety comprises the heavy chain variable region sequence of SEQ ID NO: 14 and the light chain variable region sequence of SEQ ID NO: 19.

In some aspects, the second antigen binding moiety comprises a heavy chain variable region sequence that is at least about 95%, 96%, 97%, 98%, 99% or 100% identical to the amino acid sequence of SEQ ID NO: 66 and a light chain variable region sequence that is at least about 95%, 96%, 97%, 98%, 99% or 100% identical to the amino acid sequence of SEQ ID NO: 67. In some aspects, the second antigen binding moiety comprises the heavy chain variable region sequence of SEQ ID NO: 66 and the light chain variable region sequence of SEQ ID NO: 67.

In some aspects, the T cell bispecific antibody comprises a third antigen binding moiety that binds to EpCAM and/or an Fc domain composed of a first and a second subunit, as described herein.

In preferred aspects, the T cell bispecific antibody comprises
(i) a first antigen binding moiety that binds to CD3, comprising a heavy chain variable region comprising the heavy chain CDR (HCDR) 1 of SEQ ID NO: 9, the HCDR2 of SEQ ID NO: 11, and the HCDR3 of SEQ ID NO: 12; and a light chain variable region comprising the light chain CDR (LCDR) 1 of SEQ ID NO: 16, the LCDR2 of SEQ ID NO: 17 and the LCDR3 of SEQ ID NO: 18, wherein the first antigen binding moiety is a crossover Fab molecule wherein either the variable or the constant regions, particularly the variable regions, of the Fab light chain and the Fab heavy chain are exchanged;
(ii) a second and a third antigen binding moiety that bind to EpCAM, comprising a heavy chain variable region comprising the heavy chain CDR (HCDR) 1 of SEQ ID NO: 60, the HCDR2 of SEQ ID NO: 61, and the HCDR3 of SEQ ID NO: 62; and a light chain variable region comprising the light chain CDR (LCDR) 1 of SEQ ID NO: 63, the LCDR2 of SEQ ID NO: 64 and the LCDR3 of SEQ ID NO: 65, wherein the second and third antigen binding moiety are each a Fab molecule, particularly a conventional Fab molecule;
(iii) an Fc domain composed of a first and a second subunit, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In some aspects, the first antigen binding moiety of the T cell bispecific antibody (that binds to EpCAM and CD3) is a crossover Fab molecule wherein the variable regions of the Fab light chain and the Fab heavy chain are exchanged, and wherein the second and (where present) third antigen binding moiety of the T cell bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

Particularly, in the above aspects, in the constant domain CL of the second and the third Fab molecule under (ii) the amino acid at position 124 may be substituted by lysine (K) (numbering according to Kabat) and the amino acid at position 123 may be substituted by lysine (K) or arginine (R), particularly by arginine (R) (numbering according to Kabat), and in the constant domain CH1 of the second and the third Fab molecule under (ii) the amino acid at position 147 may be substituted by glutamic acid (E) (numbering according to Kabat EU index) and the amino acid at position 213 may be substituted by glutamic acid (E) (numbering according to Kabat EU index).

In some aspects, the first antigen binding moiety of the T cell bispecific antibody (that binds to EpCAM and CD3) comprises a heavy chain variable region sequence that is at least about 95%, 96%, 97%, 98%, 99% or 100% identical to the amino acid sequence of SEQ ID NO: 14 and a light chain variable region sequence that is at least about 95%, 96%, 97%, 98%, 99% or 100% identical to the amino acid sequence of SEQ ID NO: 19. In some aspects, the first antigen binding moiety comprises the heavy chain variable region sequence of SEQ ID NO: 14 and the light chain variable region sequence of SEQ ID NO: 19.

In some aspects, the second and (where present) third antigen binding moiety of the T cell bispecific antibody (that binds to EpCAM and CD3) comprise a heavy chain variable region sequence that is at least about 95%, 96%, 97%, 98%, 99% or 100% identical to the amino acid sequence of SEQ ID NO: 66 and a light chain variable region sequence that is at least about 95%, 96%, 97%, 98%, 99% or 100% identical to the amino acid sequence of SEQ ID NO: 67. In some aspects, the second and (where present) third antigen binding moiety comprise the heavy chain variable region of SEQ ID NO: 66 and the light chain variable region of SEQ ID NO: 67.

The Fc domain according to the above aspects may incorporate, singly or in combination, all of the features described hereinabove in relation to Fc domains.

In some aspects, the Fc domain of the T cell bispecific antibody (that binds to EpCAM and CD3) comprises a modification promoting the association of the first and the second subunit of the Fc domain, and/or the Fc domain comprises one or more amino acid substitution that reduces binding to an Fc receptor and/or effector function.

In some aspects, the antigen binding moieties and the Fc region are fused to each other by peptide linkers, in particular the peptide linkers as described above.

In some aspects, the T cell bispecific antibody (that binds to EpCAM and CD3) comprises a polypeptide (particularly two polypeptides) comprising a sequence that is at least 80%, 85%, 90%, 95%, 96%, 97%, 98%, or 99% identical to the sequence of SEQ ID NO: 68, a polypeptide comprising a sequence that is at least 80%, 85%, 90%, 95%, 96%, 97%, 98%, or 99% identical to the sequence of SEQ ID NO: 69, a polypeptide comprising a sequence that is at least 80%, 85%, 90%, 95%, 96%, 97%, 98%, or 99% identical to the sequence of SEQ ID NO: 70, and a polypeptide comprising a sequence that is at least 80%, 85%, 90%, 95%, 96%, 97%, 98%, or 99% identical to the sequence of SEQ ID NO: 71. In some aspects, the T cell bispecific antibody (that binds to EpCAM and CD3) comprises a polypeptide (particularly two polypeptides) comprising the sequence of SEQ ID NO: 68, a polypeptide comprising the sequence of SEQ ID NO: 69, a polypeptide comprising the sequence of SEQ ID NO: 70, and a polypeptide comprising the sequence of SEQ ID NO: 71.

In some aspects, the target cell antigen of the T cell bispecific antibody is TYRPL.

“TYRP1” stands for tyrosinase-related protein 1 and refers to any native TYRP1 from any vertebrate source, including mammals such as primates (e.g. humans), non-human primates (e.g. cynomolgus monkeys) and rodents (e.g. mice and rats), unless otherwise indicated. The term encompasses “full-length,” unprocessed TYRP1 as well as any form of TYRP1 that results from processing in the cell. The term also encompasses naturally occurring variants of TYRP1, e.g., splice variants or allelic variants. In some aspects, TYRP1 is human TYRPL. See for the human protein UniProt (www.uniprot.org) accession no. P17643 (version 207). An exemplary sequence of human TYRP1 is given in SEQ ID NO: 7.

In some aspects, the T cell bispecific antibody comprises a first antigen binding moiety that binds to CD3, and a second antigen binding moiety that binds to TYRPL.

In some aspects, the first antigen binding moiety comprises a heavy chain variable region comprising the heavy chain CDR (HCDR) 1 of SEQ ID NO: 9, the HCDR2 of SEQ ID NO: 11, and the HCDR3 of SEQ ID NO: 12; and a light chain variable region comprising the light chain CDR (LCDR) 1 of SEQ ID NO: 16, the LCDR2 of SEQ ID NO: 17 and the LCDR3 of SEQ ID NO: 18.

In preferred aspects, the first antigen binding moiety comprises a heavy chain variable region comprising the heavy chain CDR (HCDR) 1 of SEQ ID NO: 10, the HCDR2 of SEQ ID NO: 11, and the HCDR3 of SEQ ID NO: 13; and a light chain variable region comprising the light chain CDR (LCDR) 1 of SEQ ID NO: 16, the LCDR2 of SEQ ID NO: 17 and the LCDR3 of SEQ ID NO: 18.

In some aspects, the second antigen binding moiety comprises a heavy chain variable region comprising the heavy chain CDR (HCDR) 1 of SEQ ID NO: 23, the HCDR2 of SEQ ID NO: 24, and the HCDR3 of SEQ ID NO: 25; and a light chain variable region comprising the light chain CDR (LCDR) 1 of SEQ ID NO: 27, the LCDR2 of SEQ ID NO: 28 and the LCDR3 of SEQ ID NO: 29.

In some aspects, the T cell bispecific antibody comprises
(i) a first antigen binding moiety that binds to CD3 and comprises a heavy chain variable region comprising the heavy chain CDR (HCDR) 1 of SEQ ID NO: 9, the HCDR2 of SEQ ID NO: 11, and the HCDR3 of SEQ ID NO: 12, or a heavy chain variable region comprising the HCDR1 of SEQ ID NO: 10, the HCDR2 of SEQ ID NO: 11, and the HCDR3 of SEQ ID NO: 13; and a light chain variable region comprising the light chain CDR (LCDR) 1 of SEQ ID NO: 16, the LCDR2 of SEQ ID NO: 17 and the LCDR3 of SEQ ID NO: 18; and
(ii) a second antigen binding moiety that binds to TYRP1 and comprises a heavy chain variable region comprising the heavy chain CDR (HCDR) 1 of SEQ ID NO: 23, the HCDR2 of SEQ ID NO: 24, and the HCDR3 of SEQ ID NO: 25; and a light chain variable region comprising the light chain CDR (LCDR) 1 of SEQ ID NO: 27, the LCDR2 of SEQ ID NO: 28 and the LCDR3 of SEQ ID NO: 29.

In some aspects, the first antigen binding moiety comprises a heavy chain variable region sequence that is at least about 95%, 96%, 97%, 98%, 99% or 100% identical to the amino acid sequence of SEQ ID NO: 14 or a heavy chain variable region sequence that is at least about 95%, 96%, 97%, 98%, 99% or 100% identical to the amino acid sequence of SEQ ID NO: 15, and a light chain variable region sequence that is at least about 95%, 96%, 97%, 98%, 99% or 100% identical to the amino acid sequence of SEQ ID NO: 19. In some aspects, the first antigen binding moiety comprises the heavy chain variable region sequence of SEQ ID NO: 14 or the heavy chain variable region sequence of SEQ ID NO: 15, and the light chain variable region sequence of SEQ ID NO: 19.

In some aspects, the second antigen binding moiety comprises a heavy chain variable region sequence that is at least about 95%, 96%, 97%, 98%, 99% or 100% identical to the amino acid sequence of SEQ ID NO: 26 and a light chain variable region sequence that is at least about 95%, 96%, 97%, 98%, 99% or 100% identical to the amino acid sequence of SEQ ID NO: 30. In some aspects, the second antigen binding moiety comprises the heavy chain variable region sequence of SEQ ID NO: 26 and the light chain variable region sequence of SEQ ID NO: 30.

In some aspects, the T cell bispecific antibody comprises a third antigen binding moiety that binds to TYRP1 and/or an Fc domain composed of a first and a second subunit, as described herein.

In preferred aspects, the T cell bispecific antibody comprises
(i) a first antigen binding moiety that binds to CD3 and comprises a heavy chain variable region comprising the heavy chain CDR (HCDR) 1 of SEQ ID NO: 9, the HCDR2 of SEQ ID NO: 11, and the HCDR3 of SEQ ID NO: 12, or a heavy chain variable region comprising the HCDR1 of SEQ ID NO: 10, the HCDR2 of SEQ ID NO: 11, and the HCDR3 of SEQ ID NO: 13; and a light chain variable region comprising the light chain CDR (LCDR) 1 of SEQ ID NO: 16, the LCDR2 of SEQ ID NO: 17 and the LCDR3 of SEQ ID NO: 18, wherein the first antigen binding moiety is a crossover Fab molecule wherein either the variable or the constant regions, particularly the variable regions, of the Fab light chain and the Fab heavy chain are exchanged;
(ii) a second and a third antigen binding moiety that bind to TYRP1, comprising a heavy chain variable region comprising the heavy chain CDR (HCDR) 1 of SEQ ID NO: 23, the HCDR2 of SEQ ID NO: 24, and the HCDR3 of SEQ ID NO: 25; and a light chain variable region comprising the light chain CDR (LCDR) 1 of SEQ ID NO: 27, the LCDR2 of SEQ ID NO: 28 and the LCDR3 of SEQ ID NO: 29, wherein the second and third antigen binding moiety are each a Fab molecule, particularly a conventional Fab molecule;
(iii) an Fc domain composed of a first and a second subunit, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In some aspects, the first antigen binding moiety of the T cell bispecific antibody (that binds to TYRP1 and CD3) is a crossover Fab molecule wherein the variable regions of the Fab light chain and the Fab heavy chain are exchanged, and wherein the second and (where present) third antigen binding moiety of the T cell bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

Particularly, in the above aspects, in the constant domain CL of the second and the third Fab molecule under (ii) the amino acid at position 124 may be substituted by lysine (K) (numbering according to Kabat) and the amino acid at position 123 may be substituted by lysine (K) or arginine (R), particularly by arginine (R) (numbering according to Kabat), and in the constant domain CH1 of the second and the third Fab molecule under (ii) the amino acid at position 147 may be substituted by glutamic acid (E) (numbering according to Kabat EU index) and the amino acid at position 213 may be substituted by glutamic acid (E) (numbering according to Kabat EU index).

In some aspects, the first antigen binding moiety of the T cell bispecific antibody (that binds to TYRP1 and CD3) comprises a heavy chain variable region sequence that is at least about 95%, 96%, 97%, 98%, 99% or 100% identical to the amino acid sequence of SEQ ID NO: 14 or a heavy chain variable region sequence that is at least about 95%, 96%, 97%, 98%, 99% or 100% identical to the amino acid sequence of SEQ ID NO: 15, and a light chain variable region sequence that is at least about 95%, 96%, 97%, 98%, 99% or 100% identical to the amino acid sequence of SEQ ID NO: 19. In some aspects, the first antigen binding moiety comprises the heavy chain variable region sequence of SEQ ID NO: 14 or the heavy chain variable region sequence of SEQ ID NO: 15, and the light chain variable region sequence of SEQ ID NO: 19.

In some aspects, the second and (where present) third antigen binding moiety of the T cell bispecific antibody (that binds to TYRP1 and CD3) comprise a heavy chain variable region sequence that is at least about 95%, 96%, 97%, 98%, 99% or 100% identical to the amino acid sequence of SEQ ID NO: 26 and a light chain variable region sequence that is at least about 95%, 96%, 97%, 98%, 99% or 100% identical to the amino acid sequence of SEQ ID NO: 30. In some aspects, the second and (where present) third antigen binding moiety comprise the heavy chain variable region of SEQ ID NO: 26 and the light chain variable region of SEQ ID NO: 30.

The Fc domain according to the above aspects may incorporate, singly or in combination, all of the features described hereinabove in relation to Fc domains.

In some aspects, the Fc domain of the T cell bispecific antibody (that binds to TYRP1 and CD3) comprises a modification promoting the association of the first and the second subunit of the Fc domain, and/or the Fc domain comprises one or more amino acid substitution that reduces binding to an Fc receptor and/or effector function.

In some aspects, the antigen binding moieties and the Fc region are fused to each other by peptide linkers, in particular the peptide linkers as described above.

In some aspects, the T cell bispecific antibody (that binds to TYRP1 and CD3) comprises a polypeptide (particularly two polypeptides) comprising a sequence that is at least 80%, 85%, 90%, 95%, 96%, 97%, 98%, or 99% identical to the sequence of SEQ ID NO: 33, a polypeptide comprising a sequence that is at least 80%, 85%, 90%, 95%, 96%, 97%, 98%, or 99% identical to the sequence of SEQ ID NO: 31, a polypeptide comprising a sequence that is at least 80%, 85%, 90%, 95%, 96%, 97%, 98%, or 99% identical to the sequence of SEQ ID NO: 32, and a polypeptide comprising a sequence that is at least 80%, 85%, 90%, 95%, 96%, 97%, 98%, or 99% identical to the sequence of SEQ ID NO: 34. In some aspects, the T cell bispecific antibody (that binds to TYRP1 and CD3) comprises a polypeptide (particularly two polypeptides) comprising the sequence of SEQ ID NO: 33 a polypeptide comprising the sequence of SEQ ID NO: 31, a polypeptide comprising the sequence of SEQ ID NO: 32, and a polypeptide comprising the sequence of SEQ ID NO: 34.

In preferred aspects, the T cell bispecific antibody (that binds to TYRP1 and CD3) comprises a polypeptide (particularly two polypeptides) comprising a sequence that is at least 80%, 85%, 90%, 95%, 96%, 97%, 98%, or 99% identical to the sequence of SEQ ID NO: 33, a polypeptide comprising a sequence that is at least 80%, 85%, 90%, 95%, 96%, 97%, 98%, or 99% identical to the sequence of SEQ ID NO: 31, a polypeptide comprising a sequence that is at least 80%, 85%, 90%, 95%, 96%, 97%, 98%, or 99% identical to the sequence of SEQ ID NO: 32, and a polypeptide comprising a sequence that is at least 80%, 85%, 90%, 95%, 96%, 97%, 98%, or 99% identical to the sequence of SEQ ID NO: 35. In some aspects, the T cell bispecific antibody (that binds to TYRP1 and CD3) comprises a polypeptide (particularly two polypeptides) comprising the sequence of SEQ ID NO: 33 a polypeptide comprising the sequence of SEQ ID NO: 31, a polypeptide comprising the sequence of SEQ ID NO: 32, and a polypeptide comprising the sequence of SEQ ID NO: 35.

In the following, a PD-1 axis binding antagonists that may be used as an immunotherapy in the present invention are further described. In some embodiments, the immunotherapy comprises administration of a PD-1 axis binding antagonist.

In some embodiments, the PD-1 axis binding antagonist is selected from the group consisting of a PD-1 binding antagonist, a PDL1 binding antagonist and a PDL2 binding antagonist. In some embodiments, the PD-1 axis binding antagonist is a PD-1 binding antagonist. In some embodiments, the PD-1 binding antagonist inhibits the binding of PD-1 to its ligand binding partners. In some embodiments, the PD-1 binding antagonist inhibits the binding of PD-1 to PDL1. In some embodiments, the PD-1 binding antagonist inhibits the binding of PD-1 to PDL2. In some embodiments, the PD-1 binding antagonist inhibits the binding of PD-1 to both PDL1 and PDL2. In some embodiments, the PD-1 binding antagonist is an antibody. In some embodiments, the anti-PD-1 antibody is a monoclonal antibody. In some embodiments, the anti-PD-1 antibody is an antibody fragment selected from the group consisting of Fab, Fab′-SH, Fv, scFv, and (Fab′)2 fragments. In some embodiments, the PD-1 binding antagonist is ipilimumab, nivolumab, pembrolizumab, pidilizumab, or AMP-224. In a preferred embodiment, the PD-1 binding antagonist is ipilimumab, nivolumab, or pembrolizumab.

In some embodiments, the PD-1 axis binding antagonist is a PDL1 binding antagonist. In some embodiments, the PDL1 binding antagonist inhibits the binding of PDL1 to PD-1. In some embodiments, the PDL1 binding antagonist inhibits the binding of PDL1 to B7-1. In some embodiments, the PDL1 binding antagonist inhibits the binding of PDL1 to both PD-1 and B7-1. In some embodiments, the PDL1 binding antagonist is an anti-PDL1 antibody. In some embodiments, the anti-PDL1 antibody is a monoclonal antibody. In some embodiments, the anti-PDL1 antibody is an antibody fragment selected from the group consisting of Fab, Fab′-SH, Fv, scFv, and (Fab′)₂ fragments. In some embodiments, the anti-PDL1 antibody is a humanized antibody or a human antibody. In some embodiments, the PDL1 binding antagonist is atezolizumab, durvalumab, or avelumab. In a preferred embodiment, the PDL1 binding antagonist is atezolizumab.

In some embodiments, the anti-PDL1 antibody comprises a heavy chain comprising HVR-H1 sequence of SEQ ID NO: 72, HVR-H2 sequence of SEQ ID NO: 73, and HVR-H3 sequence of SEQ ID NO: 74; and a light chain comprising HVR-L1 sequence of SEQ ID NO: 75, HVR-L2 sequence of SEQ ID NO: 76, and HVR-L3 sequence of SEQ ID NO: 77. In some embodiments, anti-PDL1 antibody comprises a heavy chain variable region comprising the amino acid sequence of SEQ ID NO: 78 and a light chain variable region comprising the amino acid sequence of SEQ ID NO: 79. In some embodiments, the anti-PDL1 antibody comprises a heavy chain comprising the amino acid sequence of SEQ ID NO: 80 and/or a light chain comprising the amino acid sequence of SEQ ID NO: 81.

In some embodiments, the PD-1 axis binding antagonist is a PDL2 binding antagonist. In some embodiments, PDL2 binding antagonist is an antibody. In some embodiments, the anti-PDL2 antibody is a monoclonal antibody. In some embodiments, the anti-PDL2 antibody is an antibody fragment selected from the group consisting of Fab, Fab′-SH, Fv, scFv, and (Fab′)₂ fragments. In some embodiments, PDL2 binding antagonist is an immunoadhesin.

In one embodiment, the cancer is selected from the group consisting of ovarian cancer, lung cancer, breast cancer, renal cancer, colorectal cancer, endometrial cancer.

In the following, adoptive cell transfer that may be used as an immunotherapy in the present invention is further described. In some embodiments, immunotherapy comprises adoptive cell transfer.

In some embodiments, adoptive cell transfer comprises administration of a chimeric antigen receptor-expressing T-cell (CAR T-cell). A skilled artisan would appreciate that CARs are a type of antigen-targeted receptor composed of intracellular T-cell signaling domains fused to extracellular tumor-binding moieties, most commonly single-chain variable fragments (scFvs) from monoclonal antibodies.

CARs directly recognize cell surface antigens, independent of MHC-mediated presentation, permitting the use of a single receptor construct specific for any given antigen in all patients. Initial CARs fused antigen-recognition domains to the CD3 activation chain of the T-cell receptor (TCR) complex. While these first-generation CARs induced T-cell effector function in vitro, they were largely limited by poor antitumor efficacy in vivo. Subsequent CAR iterations have included secondary costimulatory signals in tandem with CD3, including intracellular domains from CD28 or a variety of TNF receptor family molecules such as 4-1BB (CD137) and OX40 (CD134). Further, third generation receptors include two costimulatory signals in addition to CD3, most commonly from CD28 and 4-1BB. Second and third generation CARs dramatically improve antitumor efficacy, in some cases inducing complete remissions in patients with advanced cancer. In one embodiment, a CAR T-cell is an immunoresponsive cell modified to express CARs, which is activated when CARs bind to its antigen.

In one embodiment, a CAR T-cell is an immunoresponsive cell comprising an antigen receptor, which is activated when its receptor binds to its antigen. In one embodiment, the CAR T-cells used in the compositions and methods as disclosed herein are first generation CAR T-cells. In another embodiment, the CAR T-cells used in the compositions and methods as disclosed herein are second generation CAR T-cells. In another embodiment, the CAR T-cells used in the compositions and methods as disclosed herein are third generation CAR T-cells. In another embodiment, the CAR T-cells used in the compositions and methods as disclosed herein are fourth generation CAR T-cells.

In some embodiments, adoptive cell transfer comprises administering T-cell receptor (TCR) modified T-cells. A skilled artisan would appreciate that TCR modified T-cells can be manufactured by isolating T-cells from tumor tissue and isolating their TCRa and TCRβ chains. These TCRa and TCRβ can be cloned and transfected into T cells isolated from peripheral blood, which then express TCRa and TCRβ from T-cells recognizing the tumor. For example T cells derived from OT-1 transgenic mice used in the Examples comprise a transgenic T cell receptor designed to recognize ovalbumin residues 257-264 in the context of H2Kb. Further strategies aims to add or substitute the antigen specificity of the natural TCR complex. Different approaches to achieve this have been described. By adding antibody variable domains to the CD3 epsilon domains of the TCR, TCR complexes with a second antigen-specificity can be generated (Nolan et al., Clin. Cancer Res. (1999) 5: 3928-3941; Baeuerle et al., Nat. Comms. (2019) 10: 2087). This additional specificity can mediate peptide-human leukocyte antigen (pHLA)-independent T cell activation via the TCR complex. Another approach aims to substitute the variable alpha and beta domains of the TCR with antibody-derived variable light and variable heavy chain domains (Kuwana et al., Biochem. Biophys. Res. Commun. (1987) 149: 960-968; Liu et al., Sci. Transl. Med. (2021) 13:1-16; Mansilla-Soto et. al., Nat. Med. (2022) 28: 345-352). This combined with enzyme-mediated gene knock-outs of the endogenous TCR alpha and beta chain encoding genes, results in the loss of the natural T cell specificity and the gain of a new antigen specificity of interest.

In some embodiments, adoptive cell transfer comprises administering tumor infiltrating lymphocytes (TIL). In some embodiments, adoptive cell transfer comprises administering chimeric antigen receptor (CAR)-modified NK cells. A skilled artisan would appreciate that CAR-modified NK cells comprise NK cells isolated from the patient or commercially available NK engineered to express a CAR that recognizes a tumor-specific protein.

In some embodiments, adoptive cell transfer comprises administering dendritic cells.

In the following, cancer vaccines that may be used as an immunotherapy in the present invention are further described.

In some embodiments, the immunotherapy comprises administration of a cancer vaccine.

A skilled artisan would appreciate that a cancer vaccine exposes the immune system to a cancer-specific antigen and an adjuvant. In some embodiments, the cancer vaccine is selected from a group comprising: sipuleucel-T, GVAX, ADXS11-001, ADXS31-001, ADXS31-164, ALVAC-CEA vaccine, AC Vaccine, talimogene laherparepvec, BiovaxID, Prostvac, CDX110, CDX1307, CDX1401, CimaVax-EGF, CV9104, DNDN, NeuVax, Ae-37, GRNVAC, tarmogens, GI-4000, GI-6207, GI-6301, ImPACT Therapy, IMA901, hepcortespenlisimut-L, Stimuvax, DCVax-L, DCVax-Direct, DCVax Prostate,CBLI, Cvac, RGSH4K, SCIB1, NCT01758328, and PVX-410.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “cancer” refers to the physiological condition in mammals that is typically characterized by unregulated cell proliferation. Examples of cancer include but are not limited to, carcinoma, lymphoma, blastoma, sarcoma and leukemia. More non-limiting examples of cancers include haematological cancer such as leukemia, bladder cancer, brain cancer, head and neck cancer, pancreatic cancer, biliary cancer, thyroid cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, sarcoma, bone cancer, and kidney cancer. Other cell proliferation disorders include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases.

In some aspects, the cancer is a cancer expressing the target cell antigen of the T cell bispecific antibody.

In some aspects, the cancer is a carcinoembryonic antigen (CEA)-expressing cancer (in particular in aspects, wherein the target cell antigen of the T cell bispecific antibody is CEA). By “CEA-positive cancer” or “CEA-expressing cancer” is meant a cancer characterized by expression or overexpression of CEA on cancer cells. The expression of CEA may be determined for example by an immunohistochemistry (IHC) or flow cytometric assay. In some aspects, the cancer expresses CEA. In some aspects, the cancer expresses CEA in at least 20%, preferably at least 50% or at least 80% of tumor cells as determined by immunohistochemistry (IHC) using an antibody specific for CEA.

In some aspects, the cancer is colon cancer, lung cancer, ovarian cancer, gastric cancer, bladder cancer, pancreatic cancer, endometrial cancer, breast cancer, kidney cancer, esophageal cancer, prostate cancer, or other cancers described herein.

In particular aspects, the cancer is a cancer selected from the group consisting of colorectal cancer, lung cancer, pancreatic cancer, breast cancer, and gastric cancer. In preferred aspects, the cancer is colorectal cancer (CRC). In some aspects, the colorectal cancer is metastatic colorectal cancer (mCRC). In some aspects, the colorectal cancer is microsatellite-stable (MSS) colorectal cancer. In some aspects, the colorectal cancer is microsatellite-stable metastatic colorectal cancer (MSS mCRC).

In some aspects, the cancer is a Tyrp 1-expressing cancer (in particular in aspects, wherein the target cell antigen of the T cell bispecific antibody is Tyrp1). By “Tyrp1-positive cancer” or “Tyrp 1-expressing cancer” is meant a cancer characterized by expression or overexpression of Tyrp1 in cancer cells. The expression of Tyrp1 may be determined for example by quantitative real-time PCR (measuring Tyrp1 mRNA levels), flow cytometry, immunohistochemistry (IHC) or western blot assays. In some aspects, the cancer expresses Tyrp1. In some aspects, the cancer expresses Tyrp1 in at least 20%, preferably at least 50% or at least 80% of tumor cells as determined by immunohistochemistry (IHC) using an antibody specific for Tyrp1. In some aspects, the cancer is selected from the group consisting of kidney cancer, bladder cancer, skin cancer, lung cancer, colorectal cancer, breast cancer, brain cancer, head and neck cancer and prostate cancer.

In some aspects, the cancer is a EpCAM-expressing cancer (in particular in aspects, wherein the target cell antigen of the T cell bispecific antibody is EpCAM). By “EpCAM-positive cancer” or “EpCAM-expressing cancer” is meant a cancer characterized by expression or overexpression of EpCAM in cancer cells. The expression of EpCAM may be determined for example by quantitative real-time PCR (measuring EpCAM mRNA levels), flow cytometry, immunohistochemistry (IHC) or western blot assays. In some aspects, the cancer expresses EpCAM. In some aspects, the cancer expresses EpCAM in at least 20%, preferably at least 50% or at least 80% of tumor cells as determined by immunohistochemistry (IHC) using an antibody specific for EpCAM. In some aspects, the cancer is selected from the group consisting of colorectal, breast, gastric, prostate, ovarian, and lung cancer.

In some aspects, the cancer is a solid tumor cancer. By a “solid tumor cancer” is meant a malignancy that forms a discrete tumor mass (including also tumor metastasis) located at specific location in the patient's body, such as sarcomas or carcinomas (as opposed to e.g. blood cancers such as leukemia, which generally do not form solid tumors). Non-limiting examples of solid tumor cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, bone cancer, liver cancer and kidney cancer. Other solid tumor cancers that are contemplated in the context of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, muscles, spleen, thoracic region, and urogenital system. Also included are pre-cancerous conditions or lesions and cancer metastases.

An “individual” or “subject” herein is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). In certain aspects, the individual or subject is a human. In some aspects, the individual has a disease, particularly a disease treatable or to be treated by the immunotherapy. In some aspects, the individual has cancer, particularly a cancer treatable or to be treated by the immunotherapy. In particular, an individual herein is any single human subject eligible for treatment who is experiencing or has experienced one or more signs, symptoms, or other indicators of cancer. In some aspects, the individual has cancer or has been diagnosed with cancer, in particular any of the cancers described hereinabove. In some aspects, the individual has locally advanced or metastatic cancer or has been diagnosed with locally advanced or metastatic cancer. The individual may have been previously treated with an immunotherapy or another drug, or not so treated. In particular aspects, the patient has not been previously treated with immunotherapy. The patient may have been treated with a therapy comprising one or more drugs other than an immunotherapy before the immunotherapy is commenced.

Preferably, a T cell according to any of the aspects of the invention is a cytotoxic T cell. In some aspects the T cell is a CD4⁺ or a CD8⁺ T cell. In some aspects the T cell is a CD4⁺ T cell.

In some aspects, the treatment with or administration of the immunotherapy may result in a response in the individual. In some aspects, the response may be a complete response. In some aspects, the response may be a sustained response after cessation of the treatment. In some aspects, the response may be a complete response that is sustained after cessation of the treatment. In other aspects, the response may be a partial response. In some aspects, the response may be a partial response that is sustained after cessation of the treatment. In some aspects, the treatment with or administration of the immunotherapy and the UAP1 inhibitor may improve the response as compared to treatment with or administration of the immunotherapy alone (i.e. without the UAP1 inhibitor). In some aspects, the treatment or administration of the immunotherapy and the UAP1 inhibitor may increase response rates in a patient population, as compared to a corresponding patient population treated with the immunotherapy alone (i.e. without the UAP1 inhibitor).

The immunotherapy can be used either alone or together with other agents in a therapy. For instance, an immunotherapy may be co-administered with at least one additional therapeutic agent. In certain aspects, an additional therapeutic agent is an anti-cancer agent, e.g. a chemotherapeutic agent, an inhibitor of tumor cell proliferation, or an activator of tumor cell apoptosis.

Amino Acid Sequences

| | | SEQ |
|---|---|---|
| | Sequence | ID NO |
| Human UAP1 | MNINDLKLTLSKAGQEHLLRFWNELEEAQQVELYAELQA | 1 |
| | MNFEELNFFFQKAIEGFNQSSHQKNVDARMEPVPREVLGS | |
| | ATRDQDQLQAWESEGLFQISQNKVAVLLLAGGQGTRLGV | |
| | AYPKGMYDVGLPSRKTLFQIQAERILKLQQVAEKYYGNKC | |
| | IIPWYIMTSGRTMESTKEFFTKHKYFGLKKENVIFFQQGML | |
| | PAMSFDGKIILEEKNKVSMAPDGNGGLYRALAAQNIVEDM | |
| | EQRGIWSIHVYCVDNILVKVADPRFIGFCIQKGADCGAKVV | |
| | EKTNPTEPVGVVCRVDGVYQVVEYSEISLATAQKRSSDGR | |
| | LLFNAGNIANHFFTVPFLRDVVNVYEPQLQHHVAQKKIPY | |
| | VDTQGQLIKPDKPNGIKMEKFVFDIFQFAKKFVVYEVLRED | |
| | EFSPLKNADSQNGKDNPTTARHALMSLHHCWVLNAGGHFI | |
| | DENGSRLPAIPRSATNGKSETITADVNHNLKDANDVPIQCEI | |
| | SPLISYAGEGLESYVADKEFHAPLIIDENGVHELVKNGI | |
| UAP1 ko | ACGAACCCTACAGAACCAGTTGG | 2 |
| tracrRNA: | | |
| crRNA | | |
| Uap1-mouse | GAAAAGGTGGACGCACGAA | 3 |
| CRNA | | |
| Human CD3 | MQSGTHWRVLGLCLLSVGVWGQDGNEEMGGITQTPYKVS | 4 |
| | ISGTTVILTCPQYPGSEILWQHNDKNIGGDEDDKNIGSDEDH | |
| | LSLKEFSELEQSGYYVCYPRGSKPEDANFYLYLRARVCENC | |
| | MEMDVMSVATIVIVDICITGGLLLLVYYWSKNRKAKAKPV | |
| | TRGAGAGGRQRGQNKERPPPVPNPDYEPIRKGQRDLYSGL | |
| | NQRRI | |
| Human CEA | GLHNHHPIKPFITSNNSNPVEDEDAVALTCEPEIQNTTYLW | 5 |
| | WVNNQSLPVSPRLQLSNDNRTLTLLSVTRNDVGPYECGIQ | |
| | NELSVDHSDPVILNVLYGPDDPTISPSYTYYRPGVNLSLSCH | |
| | AASNPPAQYSWLIDGNIQQHTQELFISNITEKNSGLYTCQA | |
| | NNSASGHSRTTVKTITVSADVPKPSISSNNSKPVEDKDAVA | |
| | FHCEPEAQNTTYLWWVNGQSLPVSPRLQLSNGNRTLTLFN | |
| | VTRNDARAYVCGIQNSVSANRSDPVTLDVLYGPDTPIISPPP | |
| Human | MAPPQVLAFGLLLAAATATFAAAQEECVCENYKLAVNCF | 6 |
| EpCAM | VNNNRQCQCTSVGAQNTVICSKLAAKCLVMKAEMNGSKL | |
| | GRRAKPEGALQNNDGLYDPDCDESGLFKAKQCNGTSMCW | |
| | CVNTAGVRRTDKDTEITCSERVRTYWIIIELKHKAREKPYD | |
| | SKSLRTALQKEITTRYQLDPKFITSILYENNVITIDLVQNSSQ | |
| | KTQNDVDIADVAYYFEKDVKGESLFHSKKMDLTVNGEQL | |
| | DLDPGQTLIYYVDEKAPEFSMQGLKAGVIAVIVVVVIAVV | |
| | AGIVVLVISRKKRMAKYEKAEIKEMGEMHRELNA | |
| Human Tyrp1 | MSAPKLLSLGCIFFPLLLFQQARAQFPRQCATVEALRSGMC | 7 |
| | CPDLSPVSGPGTDRCGSSSGRGRCEAVTADSRPHSPQYPHD | |
| | GRDDREVWPLRFFNRTCHCNGNFSGHNCGTCRPGWRGAA | |
| | CDQRVLIVRRNLLDLSKEEKNHFVRALDMAKRTTHPLFVI | |
| | ATRRSEEILGPDGNTPQFENISIYNYFVWTHYYSVKKTFLG | |
| | VGQESFGEVDFSHEGPAFLTWHRYHLLRLEKDMQEMLQEP | |
| | SFSLPYWNFATGKNVCDICTDDLMGSRSNFDSTLISPNSVFS | |
| | QWRVVCDSLEDYDTLGTLCNSTEDGPIRRNPAGNVARPMV | |
| | QRLPEPQDVAQCLEVGLFDTPPFYSNSTNSFRNTVEGYSDP | |
| | TGKYDPAVRSLHNLAHLFLNGTGGQTHLSPNDPIFVLLHTF | |
| | TDAVFDEWLRRYNADISTFPLENAPIGHNRQYNMVPFWPP | |
| | VTNTEMFVTAPDNLGYTYEIQWPSREFSVPEIIAIAVVGALL | |
| | LVALIFGTASYLIRARRSMDEANQPLLTDQYQCYAEEYEKL | |
| | QNPNQSVV | |
| hIgG1 Fc | DKTHTCPPCPAPELLGGPSVFLFPPKPKDTLMISRTPEVTCV | 8 |
| region | VVDVSHEDPEVKFNWYVDGVEVHNAKTKPREEQYNSTYR | |
| | VVSVLTVLHQDWLNGKEYKCKVSNKALPAPIEKTISKAKG | |
| | QPREPQVYTLPPSRDELTKNQVSLTCLVKGFYPSDIAVEWE | |
| | SNGQPENNYKTTPPVLDSDGSFFLYSKLTVDKSRWQQGNV | |
| | FSCSVMHEALHNHYTQKSLSLSP | |
| CD3_{orig} | TYAMN | 9 |
| HCDR1 | | |
| CD3ₒₚₜ | SYAMN | 10 |
| HCDR1 | | |
| CD3_{orig} / | RIRSKYNNYATYYADSVKG | 11 |
| CD3ₒₚₜ | | |
| HCDR2 | | |
| CD3_{orig} | HGNFGNSYVSWFAY | 12 |
| HCDR3 | | |
| CD3ₒₚₜ | HTTFPSSYVSYYGY | 13 |
| HCDR3 | | |
| CD3_{orig} VH | EVQLLESGGGLVQPGGSLRLSCAASGFTFSTYAMNWVRQA | 14 |
| | PGKGLEWVSRIRSKYNNYATYYADSVKGRFTISRDDSKNT | |
| | LYLQMNSLRAEDTAVYYCVRHGNFGNSYVSWFAYWGQG | |
| | TLVTVSS | |
| CD3ₒₚₜ VH | EVQLLESGGGLVQPGGSLRLSCAASGFQFSSYAMNWVRQA | 15 |
| | PGKGLEWVSRIRSKYNNYATYYADSVKGRFTISRDDSKNT | |
| | LYLQMNSLRAEDTAVYYCVRHTTFPSSYVSYYGYWGQGT | |
| | LVTVSS | |
| CD3_{orig} / | GSSTGAVTTSNYAN | 16 |
| CD3ₒₚₜ | | |
| LCDR1 | | |
| CD3_{orig} / | GTNKRAP | 17 |
| CD3ₒₚₜ | | |
| LCDR2 | | |
| CD3_{orig} / | ALWYSNLWV | 18 |
| CD3ₒₚₜ | | |
| LCDR3 | | |
| CD3_{orig} / | QAVVTQEPSLTVSPGGTVTLTCGSSTGAVTTSNYANWVQE | 19 |
| CD3ₒₚₜ VL | KPGQAFRGLIGGTNKRAPGTPARFSGSLLGGKAALTLSGAQ | |
| | PEDEAEYYCALWYSNLWVFGGGTKLTVL | |
| CD3_{orig} IgG | EVQLLESGGGLVQPGGSLRLSCAASGFTFSTYAMNWVRQA | 20 |
| HC | PGKGLEWVSRIRSKYNNYATYYADSVKGRFTISRDDSKNT | |
| | LYLQMNSLRAEDTAVYYCVRHGNFGNSYVSWFAYWGQG | |
| | TLVTVSSASTKGPSVFPLAPSSKSTSGGTAALGCLVKDYFPE | |
| | PVTVSWNSGALTSGVHTFPAVLQSSGLYSLSSVVTVPSSSL | |
| | GTQTYICNVNHKPSNTKVDKKVEPKSCDKTHTCPPCPAPEL | |
| | LGGPSVFLFPPKPKDTLMISRTPEVTCVVVDVSHEDPEVKF | |
| | NWYVDGVEVHNAKTKPREEQYNSTYRVVSVLTVLHQDW | |
| | LNGKEYKCKVSNKALPAPIEKTISKAKGQPREPQVYTLPPS | |
| | RDELTKNQVSLTCLVKGFYPSDIAVEWESNGQPENNYKTT | |
| | PPVLDSDGSFFLYSKLTVDKSRWQQGNVFSCSVMHEALHN | |
| | HYTQKSLSLSP | |
| CD3ₒₚₜ IgG | EVQLLESGGGLVQPGGSLRLSCAASGFQFSSYAMNWVRQA | 21 |
| HC | PGKGLEWVSRIRSKYNNYATYYADSVKGRFTISRDDSKNT | |
| | LYLQMNSLRAEDTAVYYCVRHTTFPSSYVSYYGYWGQGT | |
| | LVTVSSASTKGPSVFPLAPSSKSTSGGTAALGCLVKDYFPEP | |
| | VTVSWNSGALTSGVHTFPAVLQSSGLYSLSSVVTVPSSSLG | |
| | TQTYICNVNHKPSNTKVDKKVEPKSCDKTHTCPPCPAPEA | |
| | AGGPSVFLFPPKPKDTLMISRTPEVTCVVVDVSHEDPEVKF | |
| | NWYVDGVEVHNAKTKPREEQYNSTYRVVSVLTVLHQDW | |
| | LNGKEYKCKVSNKALGAPIEKTISKAKGQPREPQVYTLPPS | |
| | RDELTKNQVSLTCLVKGFYPSDIAVEWESNGQPENNYKTT | |
| | PPVLDSDGSFFLYSKLTVDKSRWQQGNVFSCSVMHEALHN | |
| | HYTQKSLSLSP | |
| CD3_{orig} / | QAVVTQEPSLTVSPGGTVTLTCGSSTGAVTTSNYANWVQE | 22 |
| CD3ₒₚₜ IgG | KPGQAFRGLIGGTNKRAPGTPARFSGSLLGGKAALTLSGAQ | |
| LC | PEDEAEYYCALWYSNLWVFGGGTKLTVLRTVAAPSVFIFP | |
| | PSDEQLKSGTASVVCLLNNFYPREAKVQWKVDNALQSGN | |
| | SQESVTEQDSKDSTYSLSSTLTLSKADYEKHKVYACEVTH | |
| | QGLSSPVTKSFNRGEC | |
| TYRP1 | DYFLH | 23 |
| HCDR1 | | |
| TYRP1 | WINPDNGNTVYAQKFQG | 24 |
| HCDR2 | | |
| TYRP1 | RDYTYEKAALDY | 25 |
| HCDR3 | | |
| TYRP1 VH | QVQLVQSGAEVKKPGASVKVSCKASGFNIKDYFLHWVRQ | 26 |
| | APGQGLEWMGWINPDNGNTVYAQKFQGRVTMTADTSTST | |
| | VYMELSSLRSEDTAVYYCTRRDYTYEKAALDYWGQGTLV | |
| | TVSS | |
| TYRP1 | RASGNIYNYLA | 27 |
| LCDR1 | | |
| TYRP1 | DAKTLAD | 28 |
| LCDR2 | | |
| TYRP1 | QHFWSLPFT | 29 |
| LCDR3 | | |
| TYRP1 VL | DIQMTQSPSSLSASVGDRVTITCRASGNIYNYLAWYQQKPG | 30 |
| | KVPKLLIYDAKTLADGVPSRFSGSGSGTDFTLTISSLQPEDV | |
| | ATYYCQHFWSLPFTFGQGTKLEIK | |
| TYRP1 VH- | QVQLVQSGAEVKKPGASVKVSCKASGFNIKDYFLHWVRQ | 31 |
| CH1(EE) - | APGQGLEWMGWINPDNGNTVYAQKFQGRVTMTADTSTST | |
| CD3_{orig}/CD3ₒₚₜ | VYMELSSLRSEDTAVYYCTRRDYTYEKAALDYWGQGTLV | |
| VL-CH1 - Fc | TVSSASTKGPSVFPLAPSSKSTSGGTAALGCLVEDYFPEPVT | |
| (knob, | VSWNSGALTSGVHTFPAVLQSSGLYSLSSVVTVPSSSLGTQ | |
| PGLALA) | TYICNVNHKPSNTKVDEKVEPKSCDGGGGSGGGGSQAVVT | |
| | QEPSLTVSPGGTVTLTCGSSTGAVTTSNYANWVQEKPGQA | |
| | FRGLIGGTNKRAPGTPARFSGSLLGGKAALTLSGAQPEDEA | |
| | EYYCALWYSNLWVFGGGTKLTVLSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVDK | |
| | KVEPKSCDKTHTCPPCPAPEAAGGPSVFLFPPKPKDTLMISR | |
| | TPEVTCVVVDVSHEDPEVKFNWYVDGVEVHNAKTKPREE | |
| | QYNSTYRVVSVLTVLHQDWLNGKEYKCKVSNKALGAPIE | |
| | KTISKAKGQPREPQVYTLPPCRDELTKNQVSLWCLVKGFY | |
| | PSDIAVEWESNGQPENNYKTTPPVLDSDGSFFLYSKLTVDK | |
| | SRWQQGNVFSCSVMHEALHNHYTQKSLSLSP | |
| TYRP1 VH- | QVQLVQSGAEVKKPGASVKVSCKASGFNIKDYFLHWVRQ | 32 |
| CH1(EE)-Fc | APGQGLEWMGWINPDNGNTVYAQKFQGRVTMTADTSTST | |
| (hole, | VYMELSSLRSEDTAVYYCTRRDYTYEKAALDYWGQGTLV | |
| PGLALA) | TVSSASTKGPSVFPLAPSSKSTSGGTAALGCLVEDYFPEPVT | |
| | VSWNSGALTSGVHTFPAVLQSSGLYSLSSVVTVPSSSLGTQ | |
| | TYICNVNHKPSNTKVDEKVEPKSCDKTHTCPPCPAPEAAG | |
| | GPSVFLFPPKPKDTLMISRTPEVTCVVVDVSHEDPEVKFNW | |
| | YVDGVEVHNAKTKPREEQYNSTYRVVSVLTVLHQDWLNG | |
| | KEYKCKVSNKALGAPIEKTISKAKGQPREPQVCTLPPSRDE | |
| | LTKNQVSLSCAVKGFYPSDIAVEWESNGQPENNYKTTPPV | |
| | LDSDGSFFLVSKLTVDKSRWQQGNVFSCSVMHEALHNHY | |
| | TQKSLSLSP | |
| TYRP1 VL- | DIQMTQSPSSLSASVGDRVTITCRASGNIYNYLAWYQQKPG | 33 |
| CL(RK) | KVPKLLIYDAKTLADGVPSRFSGSGSGTDFTLTISSLQPEDV | |
| | ATYYCQHFWSLPFTFGQGTKLEIKRTVAAPSVFIFPPSDRKL | |
| | KSGTASVVCLLNNFYPREAKVQWKVDNALQSGNSQESVT | |
| | EQDSKDSTYSLSSTLTLSKADYEKHKVYACEVTHQGLSSPV | |
| | TKSFNRGEC | |
| CD3_{orig} VH- | EVQLLESGGGLVQPGGSLRLSCAASGFTFSTYAMNWVRQA | 34 |
| CL | PGKGLEWVSRIRSKYNNYATYYADSVKGRFTISRDDSKNT | |
| | LYLQMNSLRAEDTAVYYCVRHGNFGNSYVSWFAYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| CD3ₒₚₜ VH-CL | EVQLLESGGGLVQPGGSLRLSCAASGFQFSSYAMNWVRQA | 35 |
| | PGKGLEWVSRIRSKYNNYATYYADSVKGRFTISRDDSKNT | |
| | LYLQMNSLRAEDTAVYYCVRHTTFPSSYVSYYGYWGQGT | |
| | LVTVSSASVAAPSVFIFPPSDEQLKSGTASVVCLLNNFYPRE | |
| | AKVQWKVDNALQSGNSQESVTEQDSKDSTYSLSSTLTLSK | |
| | ADYEKHKVYACEVTHQGLSSPVTKSFNRGEC | |
| CEA HCDR1 | EFGMN | 36 |
| CEA HCDR2 | WINTKTGEATYVEEFKG | 37 |
| CEA HCDR3 | WDFAYYVEAMDY | 38 |
| CEA LCDR1 | KASAAVGTYVA | 39 |
| CEA LCDR2 | SASYRKR | 40 |
| CEA LCDR3 | HQYYTYPLFT | 41 |
| CEA VH | QVQLVQSGAEVKKPGASVKVSCKASGYTFTEFGMNWVRQ | 42 |
| | APGQGLEWMGWINTKTGEATYVEEFKGRVTFTTDTSTSTA | |
| | YMELRSLRSDDTAVYYCARWDFAYYVEAMDYWGQGTTV | |
| | TVSS | |
| CEA VL | DIQMTQSPSSLSASVGDRVTITCKASAAVGTYVAWYQQKP | 43 |
| | GKAPKLLIYSASYRKRGVPSRFSGSGSGTDFTLTISSLQPED | |
| | FATYYCHQYYTYPLFTFGQGTKLEIK | |
| CEA VL-CL | DIQMTQSPSSLSASVGDRVTITCKASAAVGTYVAWYQQKP | 44 |
| | GKAPKLLIYSASYRKRGVPSRFSGSGSGTDFTLTISSLQPED | |
| | FATYYCHQYYTYPLFTFGQGTKLEIKRTVAAPSVFIFPPSDE | |
| | QLKSGTASVVCLLNNFYPREAKVQWKVDNALQSGNSQES | |
| | VTEQDSKDSTYSLSSTLTLSKADYEKHKVYACEVTHQGLS | |
| | SPVTKSFNRGEC | |
| CEA VH- | QVQLVQSGAEVKKPGASVKVSCKASGYTFTEFGMNWVRQ | 45 |
| CH1-Fc(hole, | APGQGLEWMGWINTKTGEATYVEEFKGRVTFTTDTSTSTA | |
| PGLALA) | YMELRSLRSDDTAVYYCARWDFAYYVEAMDYWGQGTTV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQ | |
| | TYICNVNHKPSNTKVDKKVEPKSCDKTHTCPPCPAPEAAG | |
| | GPSVFLFPPKPKDTLMISRTPEVTCVVVDVSHEDPEVKFNW | |
| | YVDGVEVHNAKTKPREEQYNSTYRVVSVLTVLHQDWLNG | |
| | KEYKCKVSNKALGAPIEKTISKAKGQPREPQVCTLPPSRDE | |
| | LTKNQVSLSCAVKGFYPSDIAVEWESNGQPENNYKTTPPV | |
| | LDSDGSFFLVSKLTVDKSRWQQGNVFSCSVMHEALHNHY | |
| | TQKSLSLSPGK | |
| CD3 VL-CH1 | QAVVTQEPSLTVSPGGTVTLTCGSSTGAVTTSNYANWVQE | 46 |
| | KPGQAFRGLIGGTNKRAPGTPARFSGSLLGGKAALTLSGAQ | |
| | PEDEAEYYCALWYSNLWVFGGGTKLTVLSSASTKGPSVFP | |
| | LAPSSKSTSGGTAALGCLVKDYFPEPVTVSWNSGALTSGV | |
| | HTFPAVLQSSGLYSLSSVVTVPSSSLGTQTYICNVNHKPSNT | |
| | KVDKKVEPKSC | |
| CEA VH- | QVQLVQSGAEVKKPGASVKVSCKASGYTFTEFGMNWVRQ | 47 |
| CH1-CD3 | APGQGLEWMGWINTKTGEATYVEEFKGRVTFTTDTSTSTA | |
| VH-CL- | YMELRSLRSDDTAVYYCARWDFAYYVEAMDYWGQGTTV | |
| Fc(knob, | TVSSASTKGPSVFPLAPSSKSTSGGTAALGCLVKDYFPEPVT | |
| PGLALA) | VSWNSGALTSGVHTFPAVLQSSGLYSLSSVVTVPSSSLGTQ | |
| | TYICNVNHKPSNTKVDKKVEPKSCDGGGGSGGGGSEVQLL | |
| | ESGGGLVQPGGSLRLSCAASGFTFSTYAMNWVRQAPGKGL | |
| | EWVSRIRSKYNNYATYYADSVKGRFTISRDDSKNTLYLQM | |
| | NSLRAEDTAVYYCVRHGNFGNSYVSWFAYWGQGTLVTVS | |
| | SASVAAPSVFIFPPSDEQLKSGTASVVCLLNNFYPREAKVQ | |
| | WKVDNALQSGNSQESVTEQDSKDSTYSLSSTLTLSKADYE | |
| | KHKVYACEVTHQGLSSPVTKSFNRGECDKTHTCPPCPAPE | |
| | AAGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALGAPIEKTISKAKGQPREPQVYTLP | |
| | PCRDELTKNQVSLWCLVKGFYPSDIAVEWESNGQPENNYK | |
| | TTPPVLDSDGSFFLYSKLTVDKSRWQQGNVFSCSVMHEAL | |
| | HNHYTQKSLSLSPGK | |
| CEACAM5 | DTYMH | 48 |
| HCDR1 | | |
| CEACAM5 | RIDPANGNSKYVPKFQG | 49 |
| HCDR2 | | |
| CEACAM5 | FGYYVSDYAMAY | 50 |
| HCDR3 | | |
| CEACAM5 | RAGESVDIFGVGFLH | 51 |
| LCDR1 | | |
| CEACAM5 | RASNRAT | 52 |
| LCDR2 | | |
| CEACAM5 | QQTNEDPYT | 53 |
| LCDR3 | | |
| CEACAM5 | QVQLVQSGAEVKKPGSSVKVSCKASGFNIKDTYMHWVRQ | 54 |
| VH | APGQGLEWMGRIDPANGNSKYVPKFQGRVTITADTSTSTA | |
| | YMELSSLRSEDTAVYYCAPFGYYVSDYAMAYWGQGTLVT | |
| | VSS | |
| CEACAM5 | EIVLTQSPATLSLSPGERATLSCRAGESVDIFGVGFLHWYQ | 55 |
| VL | QKPGQAPRLLIYRASNRATGIPARFSGSGSGTDFTLTISSLEP | |
| | EDFAVYYCQQTNEDPYTFGQGTKLEIK | |
| CEACAM5 | QVQLVQSGAEVKKPGSSVKVSCKASGFNIKDTYMHWVRQ | 56 |
| VH-CH1- | APGQGLEWMGRIDPANGNSKYVPKFQGRVTITADTSTSTA | |
| Fc(hole, | YMELSSLRSEDTAVYYCAPFGYYVSDYAMAYWGQGTLVT | |
| PGLALA) | VSSASTKGPSVFPLAPSSKSTSGGTAALGCLVEDYFPEPVTV | |
| | SWNSGALTSGVHTFPAVLQSSGLYSLSSVVTVPSSSLGTQT | |
| | YICNVNHKPSNTKVDEKVEPKSCDKTHTCPPCPAPEAAGGP | |
| | SVFLFPPKPKDTLMISRTPEVTCVVVDVSHEDPEVKFNWYV | |
| | DGVEVHNAKTKPREEQYNSTYRVVSVLTVLHQDWLNGKE | |
| | YKCKVSNKALGAPIEKTISKAKGQPREPQVCTLPPSRDELT | |
| | KNQVSLSCAVKGFYPSDIAVEWESNGQPENNYKTTPPVLD | |
| | SDGSFFLVSKLTVDKSRWQQGNVFSCSVMHEALHNHYTQ | |
| | KSLSLSPGK | |
| CEACAM5 | QVQLVQSGAEVKKPGSSVKVSCKASGFNIKDTYMHWVRQ | 57 |
| VH-CH1-CD3 | APGQGLEWMGRIDPANGNSKYVPKFQGRVTITADTSTSTA | |
| VH-CL- | YMELSSLRSEDTAVYYCAPFGYYVSDYAMAYWGQGTLVT | |
| Fc(knob, | VSSASTKGPSVFPLAPSSKSTSGGTAALGCLVEDYFPEPVTV | |
| PGLALA) | SWNSGALTSGVHTFPAVLQSSGLYSLSSVVTVPSSSLGTQT | |
| | YICNVNHKPSNTKVDEKVEPKSCDGGGGSGGGGSQAVVT | |
| | QEPSLTVSPGGTVTLTCGSSTGAVTTSNYANWVQEKPGQA | |
| | FRGLIGGTNKRAPGTPARFSGSLLGGKAALTLSGAQPEDEA | |
| | EYYCALWYSNLWVFGGGTKLTVLSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVDK | |
| | KVEPKSCDKTHTCPPCPAPEAAGGPSVFLFPPKPKDTLMISR | |
| | TPEVTCVVVDVSHEDPEVKFNWYVDGVEVHNAKTKPREE | |
| | QYNSTYRVVSVLTVLHQDWLNGKEYKCKVSNKALGAPIE | |
| | KTISKAKGQPREPQVYTLPPCRDELTKNQVSLWCLVKGFY | |
| | PSDIAVEWESNGQPENNYKTTPPVLDSDGSFFLYSKLTVDK | |
| | SRWQQGNVFSCSVMHEALHNHYTQKSLSLSPGK | |
| CEACAM5 | EIVLTQSPATLSLSPGERATLSCRAGESVDIFGVGFLHWYQ | 58 |
| VL-CL | QKPGQAPRLLIYRASNRATGIPARFSGSGSGTDFTLTISSLEP | |
| | EDFAVYYCQQTNEDPYTFGQGTKLEIKRTVAAPSVFIFPPS | |
| | DRKLKSGTASVVCLLNNFYPREAKVQWKVDNALQSGNSQ | |
| | ESVTEQDSKDSTYSLSSTLTLSKADYEKHKVYACEVTHQG | |
| | LSSPVTKSFNRGEC | |
| CD3VH-CL | EVQLLESGGGLVQPGGSLRLSCAASGFTFSTYAMNWVRQA | 59 |
| | PGKGLEWVSRIRSKYNNYATYYADSVKGRFTISRDDSKNT | |
| | LYLQMNSLRAEDTAVYYCVRHGNFGNSYVSWFAYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| EpCAM | SYAIS | 60 |
| HCDR1 | | |
| EpCAM | GIIPIFGTANYAQKFQG | 61 |
| HCDR2 | | |
| EpCAM | GLLWNY | 62 |
| HCDR3 | | |
| EpCAM | RASQSVSSNLA | 63 |
| LCDR1 | | |
| EpCAM | GASTTAS | 64 |
| LCDR2 | | |
| EpCAM | QQYNNWPPAYT | 65 |
| LCDR3 | | |
| EpCAM VH | QVQLVQSGAEVKKPGSSVKVSCKASGGTFSSYAISWVRQA | 66 |
| | PGQGLEWMGGIIPIFGTANYAQKFQGRVTITADESTSTAYM | |
| | ELSSLRSEDTAVYYCARGLLWNYWGQGTLVTVSS | |
| EpCAM VL | EIVMTQSPATLSVSPGERATLSCRASQSVSSNLAWYQQKPG | 67 |
| | QAPRLIIYGASTTASGIPARFSASGSGTDFTLTISSLQSEDFA | |
| | VYYCQQYNNWPPAYTFGQGTKLEIK | |
| EpCAM VL- | EIVMTQSPATLSVSPGERATLSCRASQSVSSNLAWYQQKPG | 68 |
| CL(RK) | QAPRLIIYGASTTASGIPARFSASGSGTDFTLTISSLQSEDFA | |
| | VYYCQQYNNWPPAYTFGQGTKLEIKRTVAAPSVFIFPPSDR | |
| | KLKSGTASVVCLLNNFYPREAKVQWKVDNALQSGNSQES | |
| | VTEQDSKDSTYSLSSTLTLSKADYEKHKVYACEVTHQGLS | |
| | SPVTKSFNRGEC | |
| EpCAM VH- | QVQLVQSGAEVKKPGSSVKVSCKASGGTFSSYAISWVRQA | 69 |
| CH1(EE)- | PGQGLEWMGGIIPIFGTANYAQKFQGRVTITADESTSTAYM | |
| Fc(hole, | ELSSLRSEDTAVYYCARGLLWNYWGQGTLVTVSSASTKGP | |
| PGLALA) | SVFPLAPSSKSTSGGTAALGCLVEDYFPEPVTVSWNSGALT | |
| | SGVHTFPAVLQSSGLYSLSSVVTVPSSSLGTQTYICNVNHKP | |
| | SNTKVDEKVEPKSCDKTHTCPPCPAPEAAGGPSVFLFPPKP | |
| | KDTLMISRTPEVTCVVVDVSHEDPEVKFNWYVDGVEVHN | |
| | AKTKPREEQYNSTYRVVSVLTVLHQDWLNGKEYKCKVSN | |
| | KALGAPIEKTISKAKGQPREPQVCTLPPSRDELTKNQVSLSC | |
| | AVKGFYPSDIAVEWESNGQPENNYKTTPPVLDSDGSFFLVS | |
| | KLTVDKSRWQQGNVFSCSVMHEALHNHYTQKSLSLSPGK | |
| CD3 VH-CL | EVQLLESGGGLVQPGGSLRLSCAASGFTFSTYAMNWVRQA | 70 |
| | PGKGLEWVSRIRSKYNNYATYYADSVKGRFTISRDDSKNT | |
| | LYLQMNSLRAEDTAVYYCVRHGNFGNSYVSWFAYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| EpCAM VH- | QVQLVQSGAEVKKPGSSVKVSCKASGGTFSSYAISWVRQA | 71 |
| CH1(EE)-CD3 | PGQGLEWMGGIIPIFGTANYAQKFQGRVTITADESTSTAYM | |
| VL-CH1- | ELSSLRSEDTAVYYCARGLLWNYWGQGTLVTVSSASTKGP | |
| Fc(knob, | SVFPLAPSSKSTSGGTAALGCLVEDYFPEPVTVSWNSGALT | |
| PGLALA) | SGVHTFPAVLQSSGLYSLSSVVTVPSSSLGTQTYICNVNHKP | |
| | SNTKVDEKVEPKSCDGGGGSGGGGSQAVVTQEPSLTVSPG | |
| | GTVTLTCGSSTGAVTTSNYANWVQEKPGQAFRGLIGGTNK | |
| | RAPGTPARFSGSLLGGKAALTLSGAQPEDEAEYYCALWYS | |
| | NLWVFGGGTKLTVLSSASTKGPSVFPLAPSSKSTSGGTAAL | |
| | GCLVKDYFPEPVTVSWNSGALTSGVHTFPAVLQSSGLYSLS | |
| | SVVTVPSSSLGTQTYICNVNHKPSNTKVDKKVEPKSCDKTH | |
| | TCPPCPAPEAAGGPSVFLFPPKPKDTLMISRTPEVTCVVVDV | |
| | SHEDPEVKFNWYVDGVEVHNAKTKPREEQYNSTYRVVSV | |
| | LTVLHQDWLNGKEYKCKVSNKALGAPIEKTISKAKGQPRE | |
| | PQVYTLPPCRDELTKNQVSLWCLVKGFYPSDIAVEWESNG | |
| | QPENNYKTTPPVLDSDGSFFLYSKLTVDKSRWQQGNVFSC | |
| | SVMHEALHNHYTQKSLSLSPGK | |
| HVR-H1 | GFTFSDSWIH | 72 |
| HVR-H2 | AWISPYGGSTYYADSVKG | 73 |
| HVR-H3 | RHWPGGFDY | 74 |
| HVR-L1 | RASQDVSTAVA | 75 |
| HVR-L2 | SASFLYS | 76 |
| HVR-L3 | QQYLYHPAT | 77 |
| PDL1 VH | EVQLVESGGGLVQPGGSLRLSCAASGFTFSDSWIHWVRQA | 78 |
| | PGKGLEWVAWISPYGGSTYYADSVKGRFTISADTSKNTAY | |
| | LQMNSLRAEDTAVYYCARRHWPGGFDYWGQGTLVTVSS | |
| PDL1 VL | DIQMTQSPSSLSASVGDRVTITCRASQDVSTAVAWYQQKP | 79 |
| | GKAPKLLIYSASFLYSGVPSRFSGSGSGTDFTLTISSLQPEDF | |
| | ATYYCQQYLYHPATFGQGTKVEIKR | |
| PDL1 heavy | EVQLVESGGGLVQPGGSLRLSCAASGFTFSDSWIHWVRQA | 80 |
| chain | PGKGLEWVAWISPYGGSTYYADSVKGRFTISADTSKNTAY | |
| | LQMNSLRAEDTAVYYCARRHWPGGFDYWGQGTLVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPELLGGPSVF | |
| | LFPPKPKDTLMISRTPEVTCVVVDVSHEDPEVKFNWYVDG | |
| | VEVHNAKTKPREEQYASTYRVVSVLTVLHQDWLNGKEYK | |
| | CKVSNKALPAPIEKTISKAKGQPREPQVYTLPPSREEMTKN | |
| | QVSLTCLVKGFYPSDIAVEWESNGQPENNYKTTPPVLDSD | |
| | GSFFLYSKLTVDKSRWQQGNVFSCSVMHEALHNHYTQKS | |
| | LSLSPG | |
| PDL1 light | DIQMTQSPSSLSASVGDRVTITCRASQDVSTAVAWYQQKP | 81 |
| chain | GKAPKLLIYSASFLYSGVPSRFSGSGSGTDFTLTISSLQPEDF | |
| | ATYYCQQYLYHPATFGQGTKVEIKRTVAAPSVFIFPPSDEQ | |
| | LKSGTASVVCLLNNFYPREAKVQWKVDNALQSGNSQESV | |
| | TEQDSKDSTYSLSSTLTLSKADYEKHKVYACEVTHQGLSSP | |
| | VTKSFNRGEC | |

### BRIEF DESCRIPTION OF THE DRAWINGS

FIGS. 1A and 1B. CRISPR/Cas9 screen for novel immunomodulatory targets. FIG. 1A: Schematic representation of the screening process. FIG. 1B: Volcano plots showing enriched (LogFC>1) and depleted (LogFC<−1) in the contrast between CEA TCB and DP47 control TCB. Significant hits: P Value<0.05.

FIGS. 2A and 2B. Validation of UAP1 knockout. FIG. 2A: Western blot samples for MKN45 wt and UAP1 knockout cells probed for UAP1 expression and vinculin as loading control. FIG. 2B: LC-MS analysis of UDP-HExNAc levels in MKN45 wt and UAP1 knockout cells. Graphs show mean±SEM. P values were calculated using two-tailed unpaired Student t-test. *P<0.05, **P<0.01

FIGS. 3A, 3B, 3C and 3D. Tumor intrinsic UAP1 regulates TCB-mediated cytotoxicity in vitro. T cell-mediated killing assay. UAP1 knockout cells and wt cells for FIG. 3A MKN45 with CEA-TCB, FIG. 3B MKN45 with EpCAM-TCB, FIG. 3C A549 with CEA-TCB and FIG. 3D B16F10 with Tyrp1-TCB were used and measured was the cell count of tumor cells over time in an incucyte. Graphs show mean±SEM. P values were calculated using two-tailed paired Student t-test. *P<0.05, **P<0.01

FIG. 4. Tumor intrinsic UAP1 regulates antigen-specific T cell-mediated cytotoxicity in vitro. Tumor cell killing by antigen specific T cells. B16F10 wt and UAP1 KO cells were pulsed with SIINFEKL peptide and co-culture with OT-1 T cells. Depicted is the tumor cell count over time acquired with an incucyte machine. Graphs show mean±SEM. P values were calculated using two-tailed paired Student t-test. *P<0.05, **P<0.01

FIGS. 5A, 5B, and 5C. UAP1 expression in cancer cells affects T cell activation. Shown is the T cell activation state after a co-culture with tumor cells over a range of TCB concentrations. T cells from co-cultures with FIG. 5A MKN45 with CEA TCB, FIG. 5B MKN45 with EPCAM TCB and FIG. 5C A549 with CEA TCB were analyzed for their expression of CD25 and CD69 by FACS. Graphs show mean±SEM. P values were calculated using two-tailed paired Student t-test. *P<0.05, **P<0.01

FIG. 6. UAP1 expression in cancer cells affects T cell proliferation. T cell proliferation after co-culture of T cells with tumor cells in the presence of a TCB. MKN45 wt and UAP1 KO cells were co cultured with CFSE labeled T cells in the presence of CEA TCB. CFSE fluorescence intensity as a surrogate for proliferation was measured by FACS.

FIGS. 7A and 7B. Deletion of UAP1 sensitizes B16F10 tumors to immunotherapy. FIG. 7A: Tumor volume of B16F10 wt and UAP1 depleted cells treated with either vehicle control or Tyrp1 TCB in C57BL/6 mice immunocompetent mice. Tumor volume measured every second day by caliper. Mice per group n=10. FIG. 7B: Ratio of tumor volumes over time of TCB therapy groups and vehicle control groups for both B16F10 wt and UAP1 tumors. Two-way ANOVA statistical analysis was done with Graphpad Prism software.

FIGS. 8A, 8B, 8C, 8D, and 8E. Deletion of UAP1 sensitizes MKN45 tumors to immunotherapy. FIG. 8A: Tumor growth curves of MKN45 wt and UAP1 KO cell in stem cell humanized NSG mice (huNSG). Animals were dosed either with vehicle control, 0.2 mg/kg or 0.4 mg/kg CEA-TCB. Time point of first dosing at day 15 as indicated. Tumor volume was measured with a caliper. Mice per group (n=10). FIG. 8B: Tumor volume ratio of TCB therapy groups and vehicle control groups for every time point after the first TCB treatment. FIG. 8C: Tumor growth curves for individual mice. Two-way-ANOVA statistical analysis was done with Graphpad Prism software. FIG. 8D and FIG. 8E show the measured individual tumor volumes over time within the treatment groups corresponding to the average values shown in FIGS. 8A and FIG. 8B.

FIGS. 9A, 9B, 9C, 9D, and 9E. UAP1 inhibition with Ac4Glc2Bz in tumor cells improves T cell-mediated killing and T cell activation. FIG. 9A: Detection of UDP-HexNAc in MKN45 cell lysates after 2h treatment with Ac4Glc2Bz. FIGS. 9B and 9C: MKN45 were pre-treated with the indicated concentrations of Ac4Glc2Bz for 2 h. Afterwards, the compound was removed from the culture and T cells were added alongside TCBs at the indicated concentrations. Graphs show the % of remaining tumor cells after 105h from co-culture. DP47 TCB was used as negative control. FIGS. 9D and 9E: Flow cytometry of CD3+ T cells from the experiments in FIGS. 9B and 9C. T cell activation was assessed as previously described.

FIGS. 10A and 10B. Rescue experiment. FIG. 10A: Real-time live cell microscopy for the evaluation of T cell mediated killing. Three UAP1 isoforms were re-introduced in A549-hCEA-NLR UAP1 KO clones. Afterwards tumor cells co-culture with PBMCs from healthy donor in the presence of Cibisatamab at the indicated concentration, and incubated in the incucyte instrument. Specific lysis was calculated by counting the remaining red fluorescence intensity from tumor cells, and subsequent normalization to DP47 control TCB. Wt and UAP1 KO cells were used as reference controls. FIG. 10B: Co-cultures of tumor and T cells were performed as in FIG. 10A. After 5 days of incubation, T cell activation was assessed at FACS by measuring the % of CD25 and/or CD69 in CD3+ T cells. Graphs show mean±SEM. P values were calculated using two-tailed paired Student t-test. *P<0.05, **P<0.01

FIGS. 11A, 11B and 11C. Cytokine detection assay. MKN45-NLR wt or UAP1 KO we co-cultured in the presence of different concentrations of Cibisatamab. After 72 h, the indicated cytokines were measured via ELISA. FIG. 11A: interferon-gamma (IFN-γ); FIG. 11B: tumor necrosis factor-alpha (TNFα); FIG. 11C: Granzyme B.

FIGS. 12A and 12B. 32 days post tumor injection, scouts were taken for intra-tumor T cell activation marker analysis. Elevated expression of the T cell activation/exhaustion marker Tim3 was observed in CD8⁺ (FIG. 12A) and CD4⁺ (FIG. 12B) T cells from UAP1 KO engrafted, but not in wt engrafted mice. Data were obtained using flow cytometry. P values were calculated using two-tailed paired Student t-test. *P<0.05, **P<0.01

FIGS. 13A, 13B, 13C, 13D, and 13E. FIG. 13A: KPC WT or UAP1 KO tumor cells were engrafter in intramammary fat pads of immunocompetent C57BL/6 mice. Tumor volumes were measure 3 times/week using a calliper (n=10/group). FIG. 13B: Growth curves from single mice, relative to FIG. 13A. FIG. 13C: Statistical analysis relative to FIG. 13A. Bars show TGI at day 38. P-values were calculated using Dunn's test. FIG. 13D: 42 days post tumor injections, scouts were taken for measuring the intra-tumor abundance of in CD8⁺ and CD4⁺ T cells. Data were obtained using flow cytometry, and normalized to the tumor weight. FIG. 13E: Tumor growth curves of KPC wt or UAP1 KO tumor cells subcutaneously injected in the flank of NSG mice (n=6). Data show mean±SEM.

FIG. 14. Partial UAP1 KO results in massive hyaluronan (HA) depletion in tumor cells. HA levels in MKN45 wt or UAP1 KO tumor cells were via ELISA. Representative data of 2 independent experiments. Graphs show mean±SEM. P values were calculated using two-tailed paired Student t-test. *P<0.05, **P<0.01.

FIGS. 15A, 15B, 15C and 15D. UAP1 KO does not impair T cell functionality. FIG. 15A: T cells were polyclonally activated and subsequently electroporated with UAP1-specific gRNA:rCas9 RNPs. KO efficiency was assessed as previously described. FIG. 15B: Wt or UAP1 KO T cells were used as effector in a T cell mediated killing assay using MKN45-NLR as target cells and different concentrations of Cibisatamab as indicated. Data were measured using real-time live cell imaging and normalized to DP47 control TCB. FIG. 15C: Wt or UAP1 KO T cells were co-cultured as in (15A) Afterwards, CD25 and CD69 expression was assessed by flow cytometry. FIG. 15D: Wt or UAP1 KO T cells were activated with dynabeads at different bead:T cell ratios. Afterwards, T cell activation (CD25⁺ and/or CD69⁺) was measured by flow cytometry.

### EXAMPLES

The following are examples of methods and compositions of the invention. It is understood that various other aspects may be practiced, given the general description provided above.

### Example 1. Methods

### 1.1 CRISPR/Cas9 Screen for Novel Cancer Immunotherapy Targets

The human gastric adenocarcinoma cell line MKN45 was transduced with lentiviral particles encoding the Cas9 gene from Staphylococcus aureus (Cellecta-Cat. n. SVC9-1PS) Bat multiplicity of infection (MOI)=3, and selected with Sug/ml of Blasticidin. After selection, cells were transduced with the three-modular single-guide (sg) RNA library (Cat. n. KOHGW-M1, KOHGW-M2 and KOHGW-M3) at MOI=0.3. Cells were selected with 0.5 ug/ml of Puromycin for one week. Next, tumor cells were co-cultured with three different PBMCs from healthy donors in the presence of either CEA TCB (final concentration=400 pM) or the CEA TCB (final concentration=10 pM) or the DP47 negative control TCB (final concentration=400 pM). The library coverage was 200× on the day of the co-culture setup and the experiment was conducted using biological replicates. After 72h of incubation at 37° C. and 5% CO₂, T cells were removed and tumor cells were harvested for DNA isolation. Genomic DNA isolation was performed using the NucleoBond AXG 500 columns (Macherey-Nagel) and afterwards samples were subjected to two rounds of PCR using the NGS Prep Kit for sgRNA Libraries in pRSG16/17 (KOHGW-Cellecta-Cat.n. LNGS 120). Samples were sequenced on Illumina HiSeq4000 sequencer with dual indexing with according to the manufacturer's instructions. Data analysis was performed as follows. Demultiplexed reads were mapped to the gRNA library using STAR (v 2.7.9a) with zero mismatches allowed, sgRNA counts were estimated from bam files using Samtools. Counts were filtered and normalized using quantile normalization (using all sgRNAs regardless of their type: control or targeting). Count matrices were compared between conditions in the following three contrasts: CEA TCB vs DP47 control TCB and CEA TCB vs DP47 control TCB. Fold changes for each gene and gRNA were calculated using MAGeCK (Lit et al. 2015) and conventional differential gene expression analyses applied to sgRNA counts (voom-limma approach Law et al. 2014), coupled with enrichment test to aggregate gene level p-values (Ritchie et al. 2016). Significantly depleted and enriched genes were reported with the thresholds for p-value<0.05 and absolute logFC>1.

### 1.2.1 Generation of UAP1 Knock-Out (KO) Cells:

Generation of UAP1 KO cells was conducted by electroplating ribonuclear protein (RNP) complexes of recombinant Cas9 and UAP1-specific gRNAs using a 4D-Nucleofector (Lonza). For tumor cells, 200′000 MKN45 or A549hCEA cells were dissolved in 20 μl SF solution (Lonza) and were incubated with RNPs made of 10 ug of Cas9 (TrueCut—Thermo Scientific) and 300 pmol of 1:1 UAP1-specific tracrRNA (Alt-R® CRISPR-Cas9 tracrRNA):crRNA (ACGAACCCTACAGAACCAGTTGG) complexes. Afterward, cells were nucleofected using the nucleofection program DS137 according to the manufacturer's instructions, and incubated for at least 72h to ensure successful protein KO. UAP1 KO in T cells was obtained by pre-activating T cells from healthy donors using CD3/28/2 tetramers (Stemcell) for 24 hours, and subsequently subjecting them to nucleofection as above, using 4×10\hat{ }6 cells in 20 μl P3 solution (Lonza) and the nucleofection program EQ115. B16 mouse melanoma cells were nucleofected as for human tumor cells, using the Uap1-mouse cRNA (GAAAAGGTGGACGCACGAA) and the nucleofection program EN138.

1.2.2 Generation of Isoform-specific overexpressing cell lines

Single cell clones were obtained from A549hCEA UAP1 KO cells. Afterwards, one full UAP1 KO clone was chosen for reintroduction of different UAP1 isoforms: AGX1, AGX2 or Isoform 3. Isoform re-introduction was obtained via viral transduction.

### 1.3 Western Blot

3×10⁶ MKN45 wt or UAP1 KO were harvested and cell pellets were lysed with one volume of RIPA buffer containing protease inhibitors (Roche-Cat.n. 04693132001) for 20 min at 4° C. Afterwards, cell lysates were spun down at 10000× g for 15 min at 4° C. Supernatant was collected in a new vial and protein concentration was determined by BCA assay (Thermo Scientific-Cat. n. 23225). 30 g of protein lysates were loaded into 4-20% pre-cast protein gel (Biorad-Cat.n. 4561094) and run with the SDS-PAGE electrophoresis method (Biorad). Afterwards, proteins were transferred into PVDF membranes using the Trans-Blot Turbo Transfer System (Biorad-Cat. n. 17001919). The following primary antibodies were used for blotting: anti-human UAP1 antibody (Sigma-aldrich-Cat. n. HPA0146459), anti-Vinculin antibody (CST-Cat.n. 18799S) and anti-rabbit IgG, HRP-linked Antibody (CST-Cat. n. 7074). Membranes were incubated with Clarity Western ECL Substrate (Biorad-Cat. n. 1705060) and images were acquired using Gel Doc XR+(Biorad).

### 1.4 Detection of UDP-HexNAc by LC-MS

### Preparation of Standard Solutions:

For the preparation of calibration samples Uridine-5′-diphospho-N-acetylglucosamine sodium salt was dissolved in an 80/20 mixture of water/methanol (v/v) to a concentration of 1.00 mg/ml. A stock solution for the preparation of QC samples was prepared separately using the same procedure with separate weighing of the reference substance. Further dilutions were done in water. For calibration and quality control samples, eight and three different concentrations of Uridine-5′-diphospho-N-acetylglucosamine (UDP HExNAc) spiked into phosphate-buffered saline (PBS) were used, respectively. Two different calibration ranges were applied. For the high calibration range, the concentrations were in the range of 100 to 50000 ng/mL (100, 200, 500, 2500, 12500, 25000, 37500, and 50000 ng/mL for the calibration samples, and 300, 2500, and 37500 ng/mL for the QC samples). In the low calibration range, the concentrations were in the range of 5.00 to 5000 ng/mL (5.00, 10.0, 25.0, 100, 500, 2500, 3750, and 5000 ng/mL for the calibration samples, and 15.0, 2500, and 3750 ng/mL for the QC samples).

### Sample Preparation:

The dry cell pellets were reconstituted by addition of 500 L of PBS, followed by ultrasonification for 5 minutes. After centrifugation for 10 minutes at 4000 g and 8° C., the supernatant was collected.

### Sample Processing in the High Calibration Range:

To an aliquot of 20.0 L supernatant or calibration/QC sample, 200 L of acetonitrile, containing the internal standard (Guanosine-5-diphosphoglucose, 250 ng/mL) was added. After vortex-mixing for a few seconds, the samples were centrifuged for 10 minutes at 30000 g and 8° C. An aliquot of 200 L of the supernatant was transferred to an autosampler vial and diluted with 200 L of water. During the analysis the samples were stored at 8° C. in the autosampler tray. An aliquot of 51 of the sample was injected into the HPLC-MS/MS system.

### Sample Processing in the Low Calibration Range (Batch-4):

To an aliquot of 20.0 L supernatant or calibration/QC sample, 100 L of acetonitrile, containing the internal standard (Guanosine-5-diphosphoglucose, 10.0 ng/mL) was added. After vortex-mixing for a few seconds, the samples were centrifuged for 10 minutes at 30000 g and 8° C. An aliquot of 100 L of the supernatant was transferred to an autosampler vial. During the analysis the samples were stored at 8° C. in the autosampler tray. An aliquot of 50 L of the sample was injected into the HPLC-MS/MS system.

### Liquid Chromatography and Column Switching:

For the high calibration range the method was as follows. HPLC was performed using a binary Agilent 1290 pump (Agilent Technologies Inc, Santa Clara, CA, USA) and a PAL autosampler (CTC Analytics, Zwingen, Switzerland) equipped with a cooling stack. The mobile phase used for analytical separation consisted of water containing 10 mM ammonia acetate and 0.5% formic acid (solvent A) and acetonitrile/2-propanol 80/20 (v/v) containing 0.5% formic acid (solvent B). The HILIC column (HILICON iHilic Fusion, 5 m, 20×2.1 mm), kept at 40° C., was equilibrated with 95% solvent B at a flow rate of 0.5 mL/min. The linear gradient was increased after 0.5 minutes from 95% B to 25% B within 1.5 minutes and kept at 25% B for 0.2 minutes. The linear gradient was increased to 5% B within 0.1 minutes and kept for 1.7 minutes. From 4.1 to 5 minutes run time, the column was re-equilibrated at 95% solvent B.

For the low calibration range the method was as follows. Two-dimensional HPLC was performed using the binary pumps Agilent 1200 and Agilent 1290 pump (Agilent Technologies Inc, Santa Clara, CA, USA) and a PAL autosampler (CTC Analytics, Zwingen, Switzerland) equipped with a switching valve and a cooling stack. For trapping, the binary pump Agilent 1200 was used. The mobile phase used for trapping consisted of water containing 10 mM ammonia acetate and 0.5% formic acid (solvent A) and acetonitrile/2-propanol 80/20 (v/v) containing 0.5% formic acid (solvent B). The loading of the sample onto the trapping column was performed for 0.5 minutes using a flow rate of 1.0 mL/min and 100% solvent B. The trapping column was switched into the analytical flow after 0.5 minutes. After 3 minutes the trapping column was switched into the trapping flow for washing.

The binary mobile phase used for analytical separation consisted of water containing 10 mM ammonia acetate and 0.5% formic acid (solvent A) and acetonitrile/2-propanol 80/20 (v/v) containing 0.5% formic acid (solvent B). The HILIC column (HILICON iHilic Fusion, 5 m, 20×2.1 mm), kept at 40° C., was equilibrated with 95% solvent B at a flow rate of 0.5 mL/min. The linear gradient was increased after 0.5 minutes from 95% B to 25% B within 1.5 minutes and kept at 25% B for 0.2 minutes. The linear gradient was increased to 5% B within 0.1 minutes and kept for 1.7 minutes. From 4.1 to 5 minutes run time, the column was re-equilibrated at 95% solvent B.

### Mass Spectrometry:

A Sciex Triple Quad 6500+ instrument (AB Sciex, Concord, Canada) equipped with a Turbo V source operating in positive ion mode was used for quantitation. The temperature of the source was set to 300° C. and a spray voltage of 5500 V was used. The mass transitions were 608.1m/z to 204.0 m/z for UDP-HexNAc and 606.1 m/z to 152.2 m/z for the internal standard GDP-glucose.

### Materials:

Acetonitrile, methanol and 2-propanol were purchased from Fisher Scientific Ltd. (Loughborough, UK). Uridine-5′-diphospho-N-acetylglucosamine sodium salt, Guanosine-5-diphosphoglucose, formic acid and Phosphate-buffered saline were purchased from Sigma-Aldrich GmbH, Buchs, Switzerland.

### Determination of Protein Concentration:

The protein concentration was determined via spectrophotometric quantification after staining with bicinchoninic acid (BCA). A calibration curve was prepared with bovine serum albumin (BSA, Pierce, Illinois, USA) in the range of 50.0 to 2000 g/mL using PBS (Sigma-Aldrich GmbH, Buchs, Switzerland) as diluent.

An aliquot of 10 μL of supernatant or calibration/QC sample was mixed with 80 μL of BCA working reagent prepared by mixing 50 parts of Reagent A with 1 part of Reagent B (BCA Kit, Pierce, Illinois, USA) in a microtiter 96-well plate. After 30 minutes incubation at 37° C., the absorbance at 562 nm was measured with Spectramax i3 (Molecular Devices, CA, USA). A standard curve was prepared by plotting the calibrators absorbance vs. the concentration using a 4-parameter fit. Unknown samples concentrations were determined interpolating the absorbance to the calibration curve. Evaluation was performed with Softmax Pro GxP (Molecular Devices, CA, USA).

### 1.5 T Cell-Mediated Killing Assay

25000 MKN45-nuclight red (NLR), 20000 A549-hCEA and 10000 B16F10-NLR tumor cells (wt or UAP1 ko) were seeded in one well of a 96 well-plate. Afterwards TCBs or siinfekl were given at the indicated concentrations. Pan T cells or PBMCs, mouse splenocytes (from C57/BL6 or OT-I mice) were immediately added at the indicated E:T ratios. Co-cultures were incubated at 37° C. and 5% C02 in the Incucye S3 instrument (Sartorius) for the indicated time. Images were acquired every 3 hours. For MKN45-NLR and B16F10-NLR, T cell-mediated killing was calculated based on the red fluorescent signal coming from the tumor cells. All values were first transformed to fold-change to time 0, and % specific lysis was calculated as follows:

100−[(Red fluorescent counts TCB/Red fluorescent counts control TCB)×100]

For A549-hCEA, the cytotox-green reagent (sartorius) was added to the co-culture at 1:16000 dilution and T-cell mediated killing was assesses by measuring green fluorescent count in each well. All values were first transformed to fold-change to time 0, and then % normalized green signal was calculated as follows: [(Green fluorescent counts TCB/Green fluorescent counts control TCB)×100]. For the antigen-specific experiment, B16F10-NLR wt and UAP1 KO cells were pulsed with SIINFEKL peptide and co-culture with OT-1 T cells. T-cell mediated killing was measured as above.

### 1.6 Flow Cytometry

T cell-mediated killing assay was performed as described above. At the end of the experiment, T cells were collected from the co-culture plates and subjected to immunofluorescence for the following markers: CD3, CD8, CD25, CD69 and Near-IR live/dead staining (Thermo Scientific). Cells were gated on live CD3⁺ cells and % Activated cells was calculated as such: 100-(% CD25⁺+% CD69⁺ cells). For the proliferation assay, T cells were pre-stained with CFSE (Biolegend) according to manufacturer's instructions, and then used in a T cell-mediated killing assay with either MKN45-NLR wt or MKN45-NLR KO cells for 4 days. Afterwards, cells were harvested and CFSE was assessed by FACS.

### 1.7 In Vivo Studies

### Animals:

Immunocompetent C57BL/6J and NOD.Cg-PrkdcscidIL-2rgtm1Wjl/SzJ (NSG) mice were obtained from Charles River Laboratories. All animal studies were performed under local Swiss government ethical approval and regulations (license ZH183/2020) according to international FELASA and national GV-Solas and TierSchG guidelines, respectively. After arrival, animals were maintained for one week to get accustomed to the new environment and for observation. Mice were maintained under specific-pathogen-free conditions with daily cycles of 12 h light/12 h darkness. Continuous health monitoring was carried out on a daily basis. Mice were housed in standardized conditions, and they had access to water and food ad libitum. For humanization, NSG mice were injected with Busulfan (15 mg/kg) followed 24 hours later by an injection of human CD34+ cord blood cells (1×10⁵ per mouse; STEMCELL Technologies) as described earlier (PMID: 33330050). Before tumor cell inoculation, humanized mice were screened for human T-cell frequencies by flow cytometry and only mice with more than 20% huCD45+ cells were randomized into different treatment groups.

### Study Design:

B16F10 and MKN45 wtl and UAP1 KO cells were trypsinized, washed and resuspended using a 1:1 mixture of tissue culture medium and Matrigel in a total volume of 100 μl. B16F10 (0.5×10⁶) and MKN45 (1×10⁶) cells were injected subcutaneously (s.c.) in the flank of the mice. Tumors were measured twice weekly and tumor volume (mm3) was determined using the formula 0.5×length×width2. MKN45 tumor bearing mice with tumor sizes of 100-200 mm3 were randomized into 4 groups: 1) NSG (n=6), 2) humanized mice huNSG (n=11) receiving intravenous (i.v.) injection of vehicle (n=11), 3-4) huNSG (n=12) receiving intravenous (i.v.) injection of 0.2 and 0.4 mg/kg of CEA-TCB once per week. B16F10 tumor bearing mice with tumor sizes of 100-150 mm3 were randomized into three groups: 1) NSG (n=6), 2) C57BL/6J (n=10) receiving intravenous (i.v.) injection of vehicle, 3) C57BL/6J (n=10) receiving intravenous (i.v.) injection of Tyrp1-TCB 5 mg/kg once per week.

All mice were injected i.v. with 200 μl of the appropriate solution. The mice in the vehicle group were injected i.v. with Histidine buffer (20 mM Histidine, 140 mM NaCl, pH 6.0) and the treatment group with the antibody diluted with Histidine buffer to a volume of 200 μl.

For some examples, KPC (0.3×106) wt and KO cells were injected into intramamammary fat pads of C57/BL6 mice similar as described above for B16F10 and MKN45 cell.

### Statistical Analysis:

The results are presented as mean±standard error of the mean (SEM). Statistical analyses were performed using GraphPad Prism software v.7.04 (GraphPad Software Inc.). 2-way Anova test was used. A P value of less than 0.05 was considered statistically significant.

Tumor growth inhibition (TGI) for each group and time point was calculated as

100−Average(TVtreatment[day.x]−TVtreatment[baseline])/Average(TVreference[day.x]−TVref erence[baseline]

### Example 2. Cancer Immunotherapy CRISPR Screen in Tumor Cells

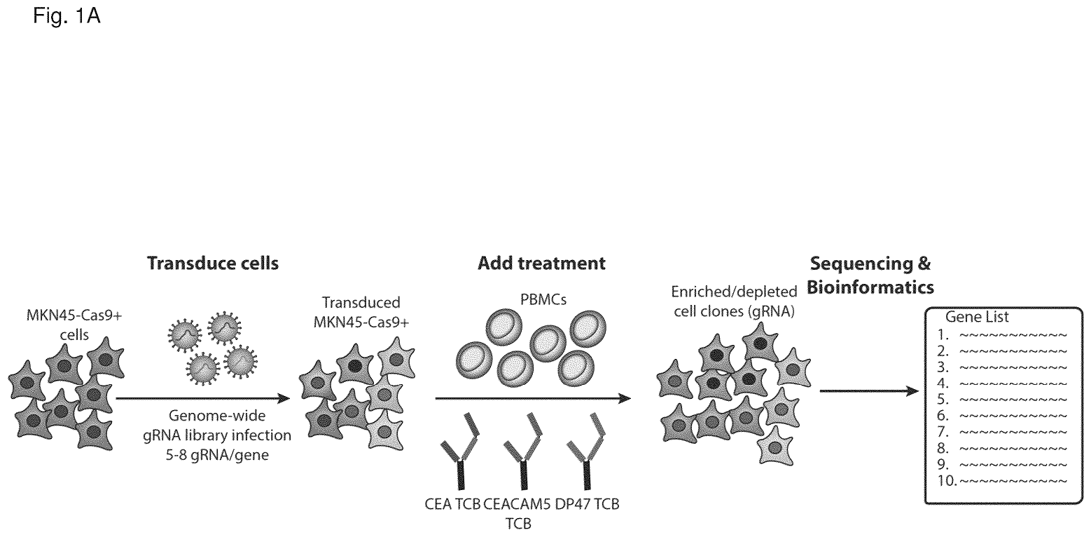

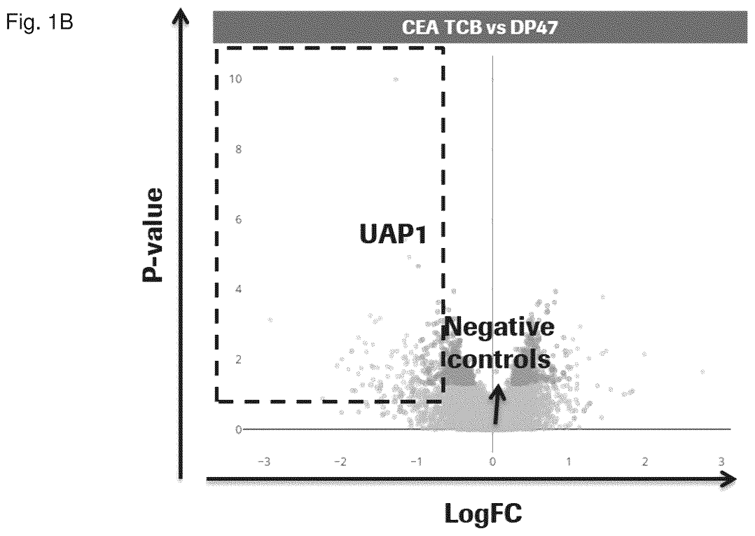

A knockout CRISPR screen in the context of an immune therapy was established to identify resistance mechanisms in cancer cells towards immune cell attack (FIGS. 1A and 1B). In brief, the human gastric adenoca/rcinoma cell line MKN45 was transduced with Cas9 and a genome wide library of sgRNAs. The selected cells were co-cultured with isolated PBMCs from healthy donors in the presence of a T cell engaging antibody targeting the tumor antigen CEACAM5 or a mock control antibody for 4 days. After this period, T cells were washed away and remaining tumor cells were collected for DNA isolation. The composition of remaining sgRNAs in the cell pool was detected by next generation sequencing. sgRNA that were significantly depleted in the group with Cibisatamab (CEA TCB) compared to control TCB antibody were followed up in dedicated validation experiments. UAP1 was identified as one of the top hits from this screen.

### Example 3. Knockout Generation and Validation In Vitro Models

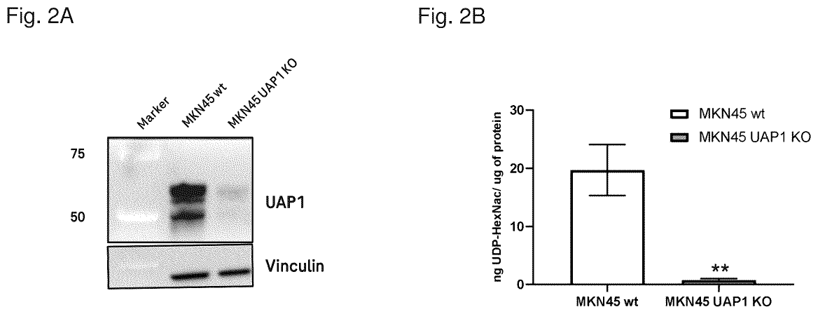

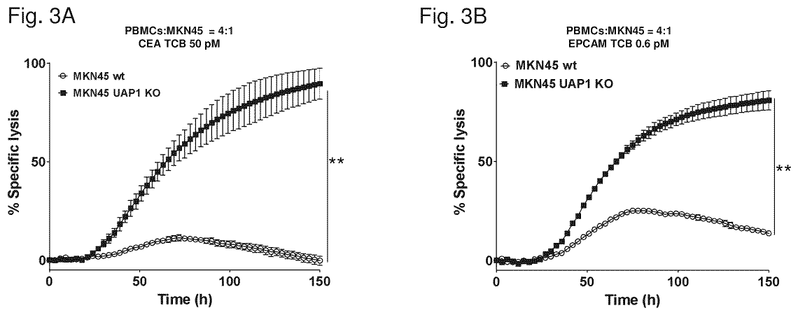

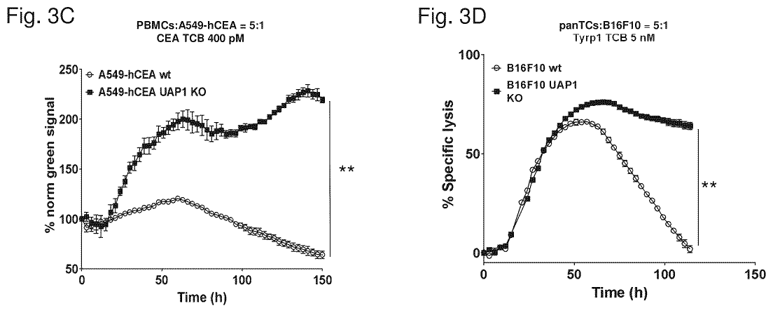

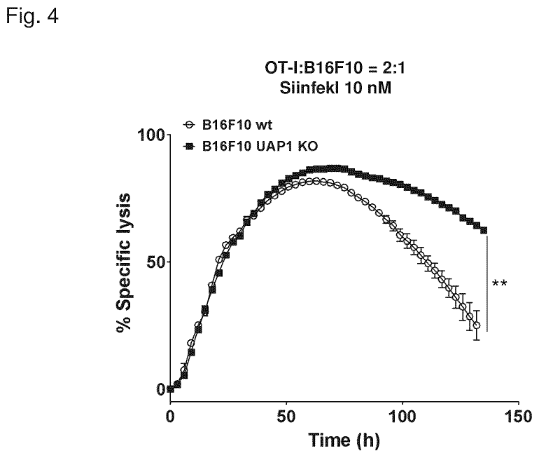

To validate UAP1, MKN45-NLR cells were subjected to UAP1 KO as described above. The knockout was confirmed on protein level (FIG. 2A) and by the reduction of the enzymatic product UDP-HexNAc in cellular lysates (FIG. 2B). Next, the KO cell line was subjected to the same killing assay conditions as described for the screen. MKN45 cells deficient for UAP1 were killed more efficiently compared to wt cells, suggesting a higher susceptibility to TCB-mediated T cell cytotoxicity (FIG. 3A). The same effect was observed when using a TCB targeting EpCAM instead of CEACAM5 (FIG. 3B). Switching cell lines to the human lung adenocarcinoma cell line A549 resulted in the same phenotype. A549 UAP1 KO cells were more efficiently killed in a CEA-TCB dependent manner than A549 wt cells (FIG. 3C). In line with the results, murine B16F10 melanoma cells deficient for UAP1 were more efficiently killed by T cells when engaged with Tyrp1-TCB compared to B16F10 wt cells (FIG. 3D). The B16F10 model can be used in a antigen specific setting, mimicking a endogenous immune reaction of T cells to an immunogenic antigen in the context of MHC-I. B16F10 cells wt or deficient for UAP1 were pulsed with the model antigen peptide SIINFEKL and co-cultured with T cells derived from a OT-1 transgenic mouse. These T cells carry a transgenic T cell receptor recognizing the H2kb-SIINFEKL complex. Similar to the TCB-mediated killing assays, UAP1 deficient cells were more susceptible to T cell cytotoxicity (FIG. 4).

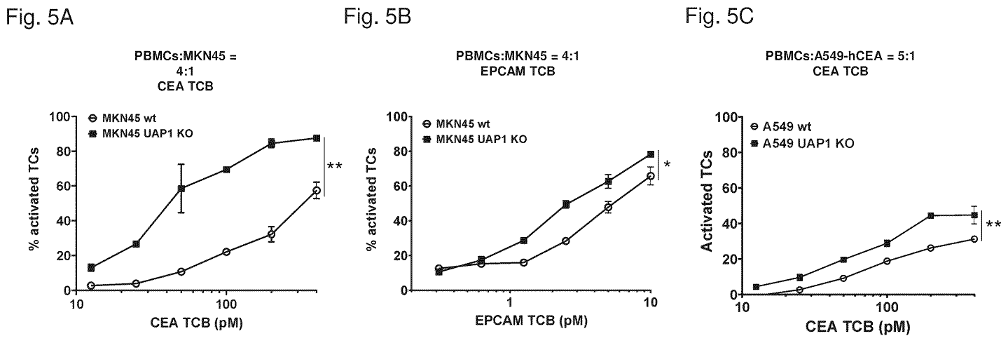

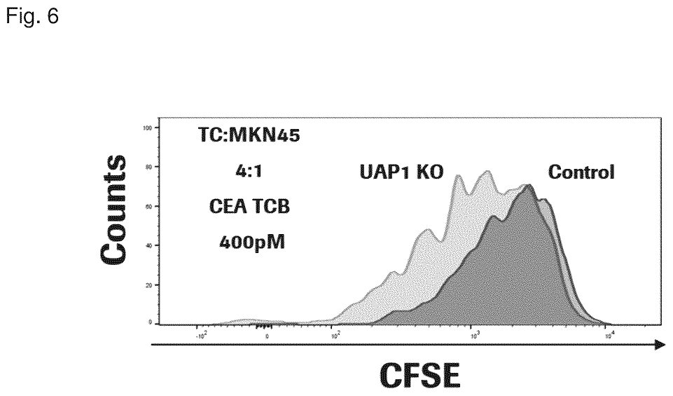

Next, the activation state of T cells co-cultured with wt or UAP1 KO cells was assessed. T cells co-cultured with UAP1 KO cancer cells expressed higher levels of early activation markers CD25 and CD69 compared to wt at similar concentrations of TCB for all cells three cell lines tested (FIG. 5A-C). In line with these results, T cells co-cultured with UAP1 KO had a higher proliferation rate than their wt counterparts (FIG. 6).

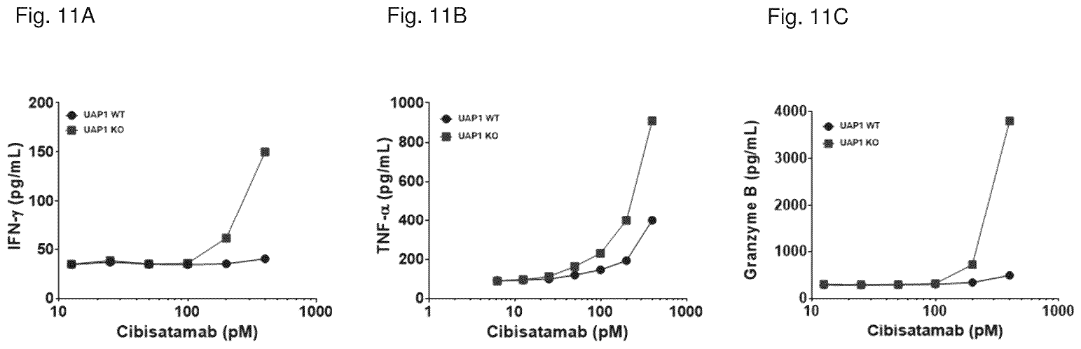

It was additionally found that T cell co-cultured with UAP1 KO secreted more IFN-γ, TNF-α and granzyme B (FIGS. 11A-11C).

These data suggest that UAP1 expression in cancer cells serves as a resistance mechanism to an anti-cancer immune response triggered either by a T cell engaging antibody or by an endogenous immune response.

### Example 4. Validation in Tumor Bearing Animal Models

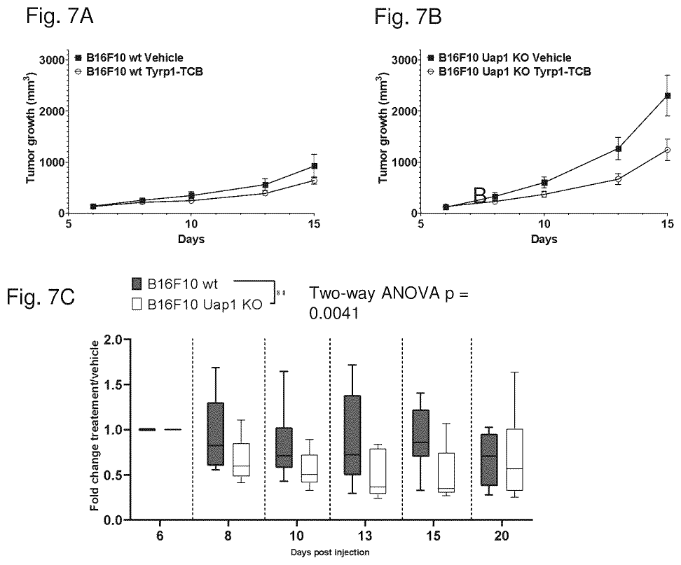

Next, the role of UAP1 was explored in tumor bearing mouse models in the context of a TCB-induced immune response. First, B16F10 melanoma cells wt or deficient for UAP1 were transplanted into C57BL/6 mice and treated either with a vehicle control a TCB targeting melanoma antigen Tyrp1 while the tumor volume was followed over time. B16F10 UAP1 KO cells grew much slower relative to their vehicle group compared to their wt counterparts (FIG. 7A). The ratio of TCB treated tumor volume over vehicle treated tumor volume showed a significant decrease of UAP1 KO cells compared to wt cells (FIG. 7B).

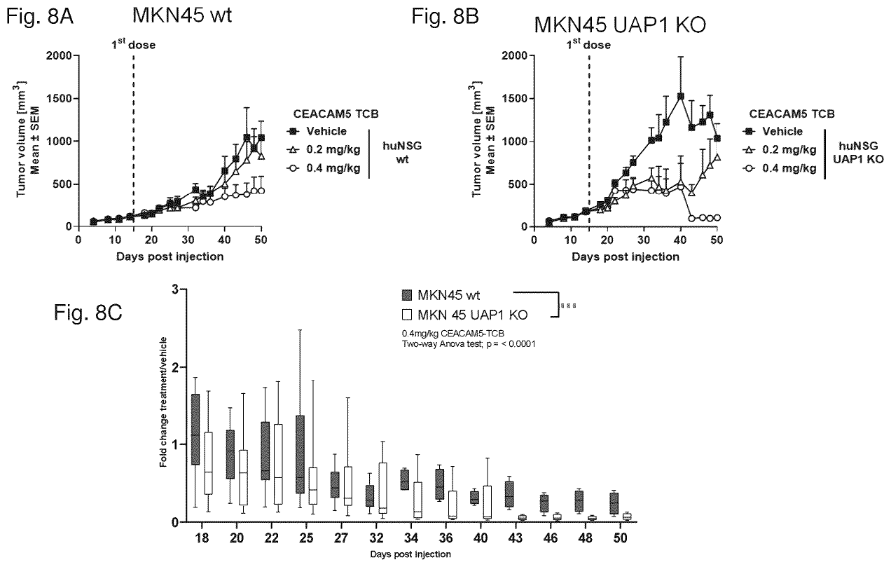

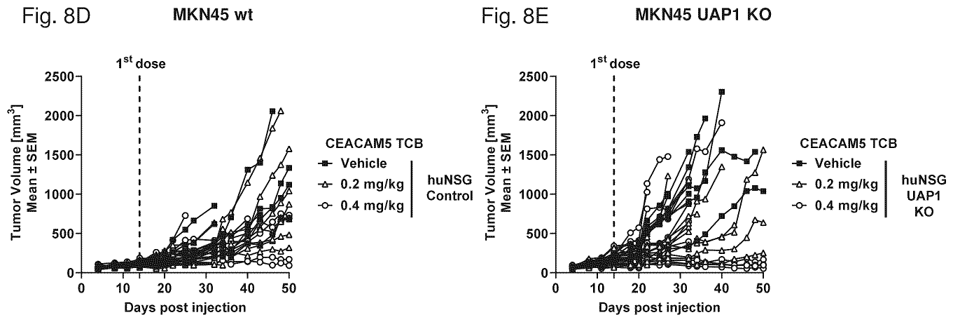

Stem cell humanized mouse models provide an elegant way to mimic parts of the human immune system in a living model organism. In brief, bone marrow depleted NSG mice underwent a cord blood derived hematopoietic stem cell transplantation. The transplanted stem cells reconstitute the bone marrow and develop into human T cells and B cells. Combining humanized mice together with a xenograft, the human gastric adenocarcinoma cell line MKN45, offers a model to test the effect of UAP1 expression on tumor growth in vivo. Consequently, MKN45 wt or UAP1 KO cells were transplanted into stem cell humanized NSG mice and treated either with a vehicle control, a low dose TCB treatment or a high dose TCB treatment targeting the tumor antigen CEACAM5. Longitudinal tumor volume measurements highlight a better tumor control of UAP1 KO cells by the TCB treatment relative to the vehicle group compared to the wt cells for both low and high dose groups (FIG. 8A and FIG. 8C). Quantifying the ratio of treatment to vehicle tumor volume for the high dose group gave a significantly decreased tumor volume when UAP1 was knocked out compared to wt (FIG. 8B). FIG. 8D and FIG. 8E show the measured individual tumor volumes over time within the treatment groups corresponding to the average values shown in FIG. 8A and FIG. 8B.

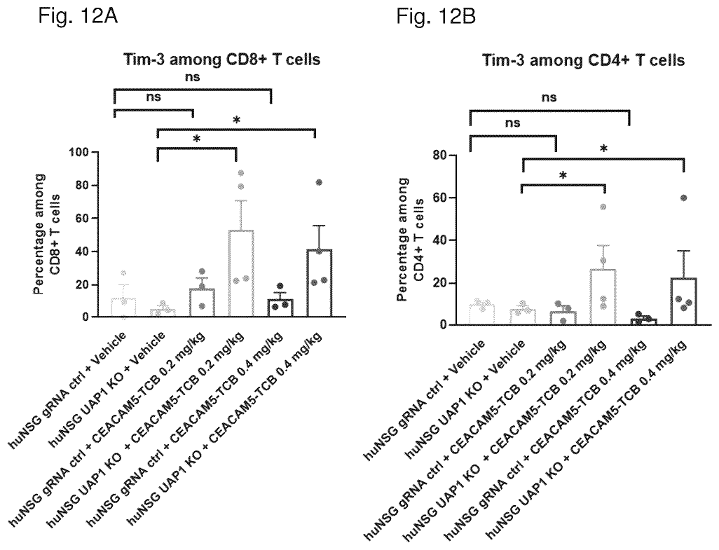

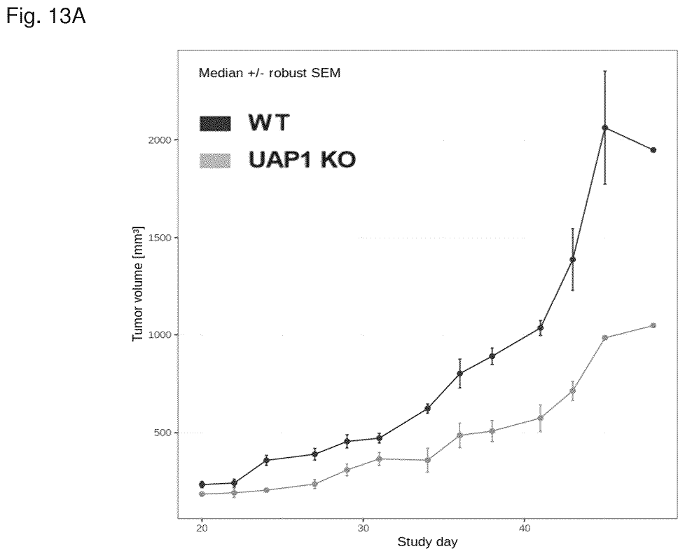

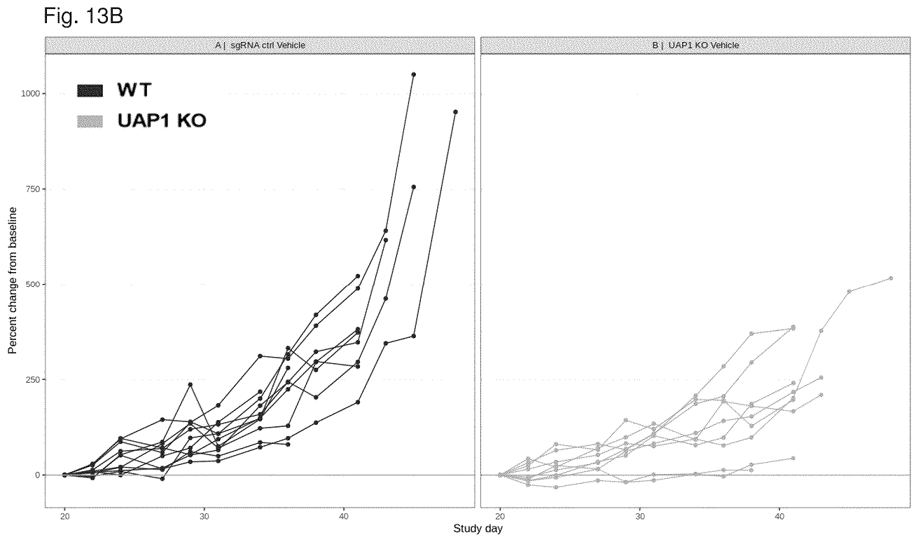

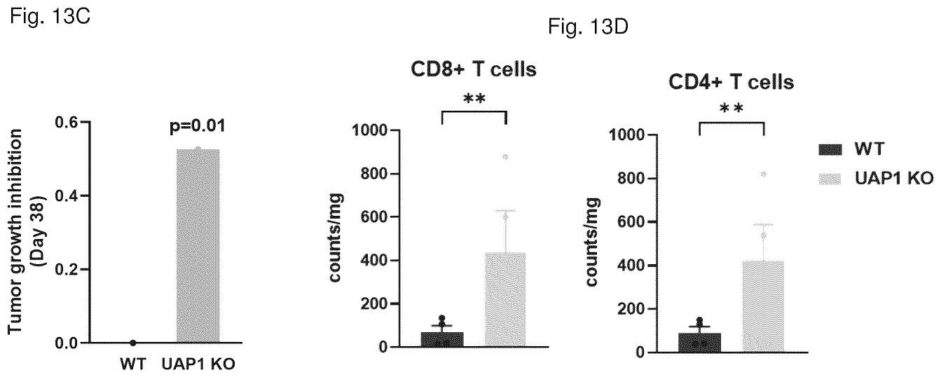

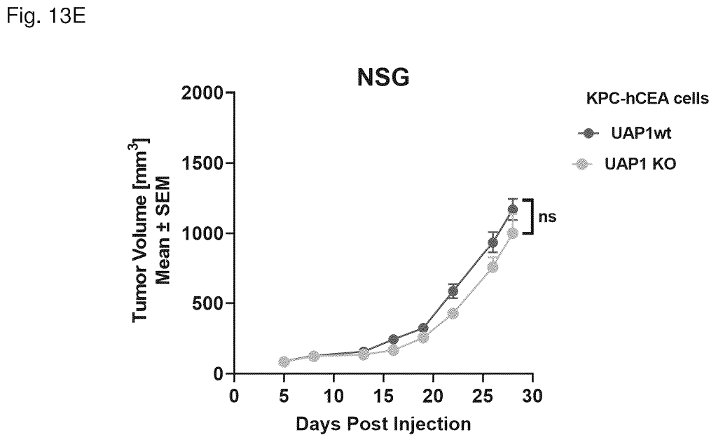

Furthermore, increased expression of the activation/exhaustion marker was observed in UAP1 KO groups in the presence of CEACAM5xCD5 TCB but not in wt cells, indicating a higher activation of T cells in the UAP1 KO tumors compared to control (FIGS. 12A and 12B). To prove standalone efficacy, UAP1 was knockedout in KPC pancreatic ductal adenocarcinoma cell lines and injected into the intramammary fat pads of C57/BL6 mice in the absence of immunotherapy. Data showed strong increased of tumor growth inhibition in UAP1 KO groups compared to wt (FIGS. 13A-13C). Additionally UAP1 KO tumors showed increase infiltration of both CD8+ and CD4+ T cells (FIG. 13D). To confirm that the observed effect was due to the involvement of the endogenous mouse immune system, the experiment was repeated in NSG immunodeficient mice. In this mouse model, UAP1 KO did not improve tumor growth inhibition (FIG. 13E).

Taken together, these data are in line with the results from the screen and the in vitro validation experiments. The effect of an increased anti-tumor immunity when UAP1 is knocked out in tumor cells was translated into in vivo settings for both, syngeneic and stem cell humanized xenograft models.

### Example 5. Pharmacological Inhibition of UAP1 in Tumor Cells Recapitulates the Effects Observed with the Genetic Knock-Out

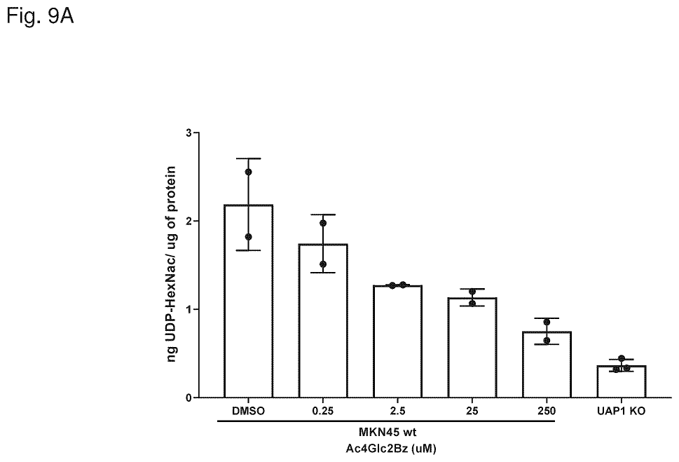

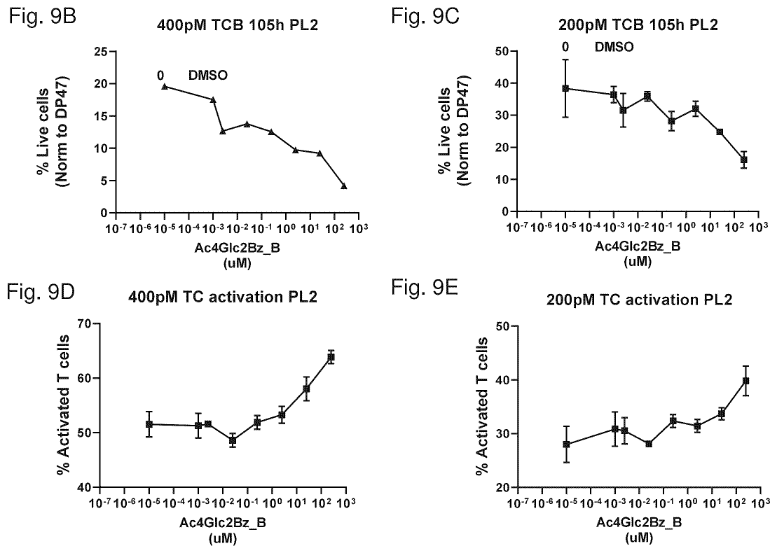

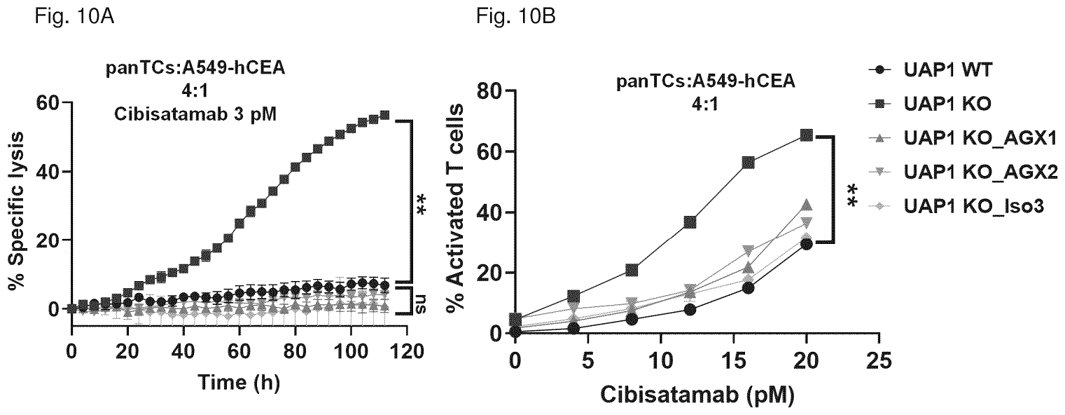

To assess the effect of UAP1 pharmacological inhibition in modulating T cell functions, MKN45 were pre-treated with the Ac4Glc2Bz tool compound for 2h. LC-MS for the detection of UDP-HexNAc confirmed dose-dependent inhibition of UAP1 (FIG. 9A). Afterwards, UAP1-inhibited MKN45 cells were subjected to co-culture with T cells and TCBs. Coherently with the UAP1 KO experiments, UAP1 inhibition led to both increased T cell mediated killing (FIGS. 9B and 9C) and T cell activation (FIGS. 9D and 9E).

### Example 6. UAP1 is a Positive Regulator of Hyaluronan (HA), an Immunosuppressive Glycosaminoglycan (GAG)

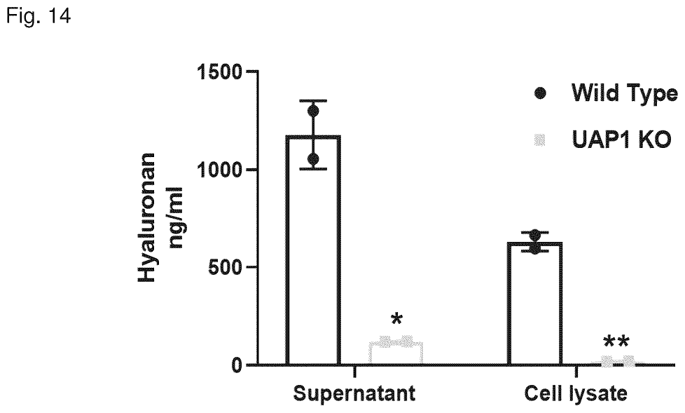

Given the pivotal role of UAP1 in regulating several glycosylation-related processes in cells, expression of HA was assessed, as this GAG was reported to confer immune resistance to tumor cells. UAP1 KO resulted in significant decrease of HA both in the supernatant and in the cell lysates of M KN45 tumor cells (FIG. 14).

### Example 7. UAP1 Depletion does not Impair T Cell Functionality

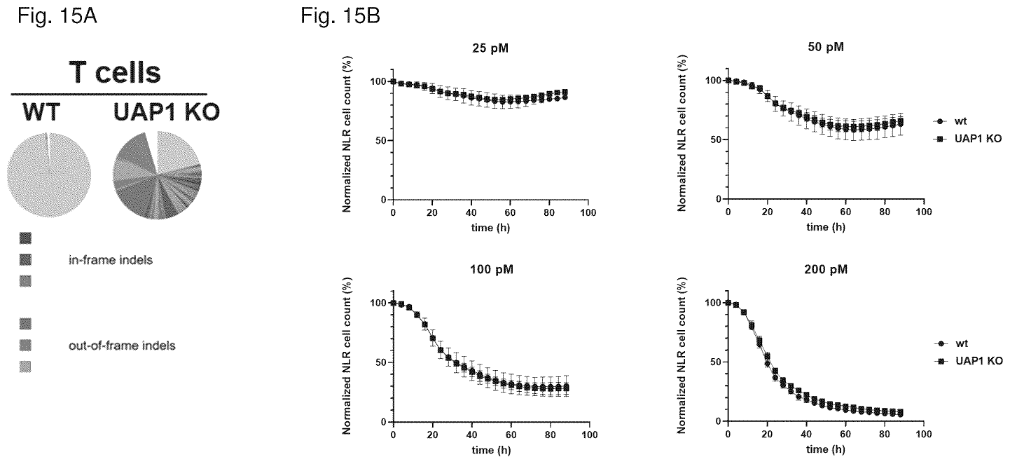

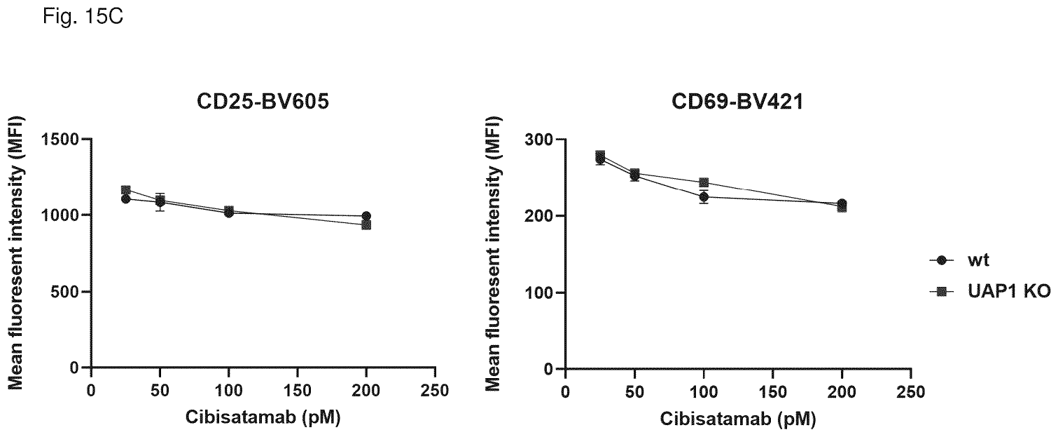

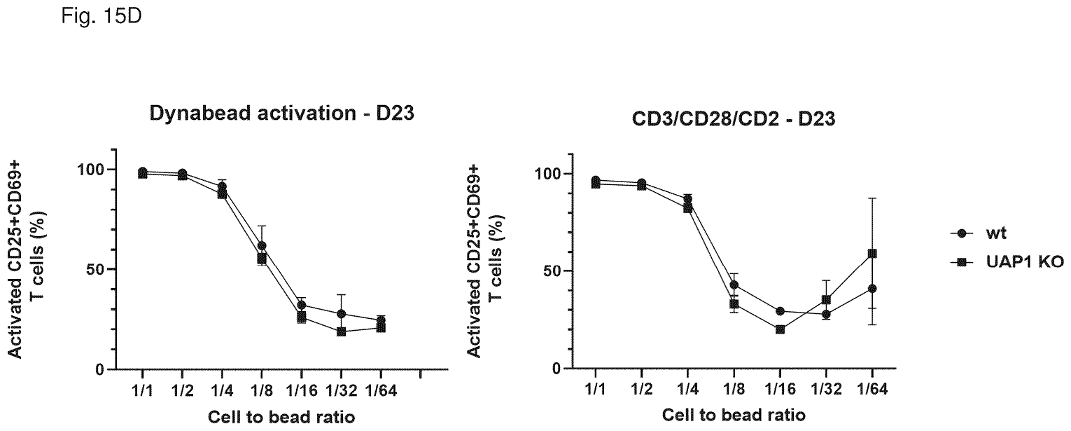

To prove that a potential UAP1 inhibitor compound would only affect tumor cells, and not T cell functionality, UAP1 was depleted on T cells and its impact on their functionality was assessed by different means. Notably UAP1 KO did neither impair T cell mediated killing, nor T cell activation (FIGS. 15A-15D).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. (canceled)

2. A method for treatment of cancer in an individual,
wherein said method comprises:
(a) administration of a therapeutically effective dose of UDP-N-acetylhexosamine pyrophosphorylase (UAP1) inhibitor to the individual, and
(b) administration of a therapeutically effective dose of an immunotherapy to the individual.

3. (canceled)

4. (canceled)

5. The method of claim 2, wherein the immunotherapy comprises adoptive cell transfer, administration of monoclonal antibodies, administration of cytokines, administration of a cancer vaccine, T cell engaging therapies, administration of PD-1 axis binding antagonists or any combination thereof.

6. The method of claim 2, wherein administration of the UAP1 inhibitor causes
(i) an increase of the activity of the immunotherapy,
(ii) an increase of the activation of T cells induced by the immunotherapy,
(iii) an increase of the proliferation of T cells induced by the immunotherapy,
(iv) an increase of the cytotoxic activity of T cells induced by the immunotherapy,
(v) an increase of T cell receptor signaling in T cells induced by the immunotherapy,
(vi) an increase of early activation markers CD25 and/or CD69 in T cells (induced by the immunotherapy),
(vii) an increase of cytokine secretion by T cells induced by the immunotherapy, wherein said cytokine is one or more of IL-2, TNF-α, and/or IFN-γ; and/or
(viii) an increase of cytolytic effector molecule secretion by T cells induced by the immunotherapy, particularly wherein the cytolytic effector molecule is granzyme-B or perforin;
wherein said T cells are CD8+ T cells or CD4+ cells.

7. The method of claim 6, wherein administration of the UAP1 inhibitor causes increase of the level of one of more cytokine in the individual, wherein said one or more cytokine is IL-2, TNF-α, and/or IFN-γ.

8. The method of claim 2, wherein administration of the UAP1 inhibitor is (i) before, concurrent to, or after the administration of the immunotherapy, (ii) intermittently or continuously, and/or (iii) oral.

9-12. (canceled)

13. The method of claim 2, wherein administration of the UAP1 inhibitor is associated with the first administration of the immunotherapy.

14. (canceled)

15. (canceled)

16. The method of claim 2, wherein the immunotherapy is a T cell bispecific antibody, wherein the T cell bispecific antibody comprises an antigen binding moiety that binds to CD3 and an antigen binding moiety that binds to a target cell antigen.

17. (canceled)

18. The method of claim 16, wherein the target cell antigen is carcinoembryonic antigen (CEA); and wherein the cancer is a carcinoembryonic antigen (CEA)-expressing cancer.

19. The method of claim 18, wherein the T cell bispecific antibody comprises:
(i) a first antigen binding moiety that binds to CD3 and comprises a heavy chain variable region comprising the heavy chain CDR (HCDR) 1 of SEQ ID NO: 9, the HCDR2 of SEQ ID NO: 11, and the HCDR3 of SEQ ID NO: 12; and a light chain variable region comprising the light chain CDR (LCDR) 1 of SEQ ID NO: 16, the LCDR2 of SEQ ID NO: 17 and the LCDR3 of SEQ ID NO: 18; and
(ii) a second antigen binding moiety that binds to CEA and comprises a heavy chain variable region comprising the heavy chain CDR (HCDR) 1 of SEQ ID NO: 36, the HCDR2 of SEQ ID NO: 37, and the HCDR3 of SEQ ID NO: 38; and a light chain variable region comprising the light chain CDR (LCDR) 1 of SEQ ID NO: 39, the LCDR2 of SEQ ID NO: 40 and the LCDR3 of SEQ ID NO: 41.

20. The method of claim 18, wherein the T cell bispecific antibody comprises a third antigen binding moiety that binds to CEA and/or an Fc domain composed of a first and a second subunit.

21. The method of claim 18, wherein the T cell bispecific antibody comprises;
(i) a first antigen binding moiety that binds to CD3, comprising a heavy chain variable region comprising the heavy chain CDR (HCDR) 1 of SEQ ID NO: 9, the HCDR2 of SEQ ID NO: 11, and the HCDR3 of SEQ ID NO: 12; and a light chain variable region comprising the light chain CDR (LCDR) 1 of SEQ ID NO: 16, the LCDR2 of SEQ ID NO: 17 and the LCDR3 of SEQ ID NO: 18, wherein the first antigen binding moiety is a crossover Fab molecule wherein either the variable or the constant regions of the Fab light chain and the Fab heavy chain are exchanged;
(ii) a second and a third antigen binding moiety that bind to CEA, comprising a heavy chain variable region comprising the heavy chain CDR (HCDR) 1 of SEQ ID NO: 36, the HCDR2 of SEQ ID NO: 37, and the HCDR3 of SEQ ID NO: 38; and a light chain variable region comprising the light chain CDR (LCDR) 1 of SEQ ID NO: 39, the LCDR2 of SEQ ID NO: 40 and the LCDR3 of SEQ ID NO: 41; wherein the second and third antigen binding moiety are each a Fab molecule, particularly a conventional Fab molecule;
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

22. The method of claim 19, wherein the first antigen binding moiety of the T cell bispecific antibody comprises a heavy chain variable region sequence that is at least 98% identical to the amino acid sequence of SEQ ID NO: 14 and a light chain variable region sequence that is at least identical to the amino acid sequence of SEQ ID NO: 19, and/or the second and (where present) third antigen binding moiety of the T cell bispecific antibody comprise a heavy chain variable region sequence that is at least 98% identical to the amino acid sequence of SEQ ID NO: 14 and a light chain variable region sequence that is at least 98% identical to the amino acid sequence of SEQ ID NO: 19.

23. (canceled)

24. The method of claim 2, wherein the immunotherapy is cibisatamab.

25. The method of claim 16, wherein the target cell antigen is epithelial cellular adhesion molecule (EpCAM); and wherein the cancer is an epithelial cellular adhesion molecule (EpCAM)-expressing cancer.

26. The method of claim 25, wherein the T cell bispecific antibody comprises:
(i) a first antigen binding moiety that binds to CD3 and comprises a heavy chain variable region comprising the heavy chain CDR (HCDR) 1 of SEQ ID NO: 9, the HCDR2 of SEQ ID NO: 11, and the HCDR3 of SEQ ID NO: 12; and a light chain variable region comprising the light chain CDR (LCDR) 1 of SEQ ID NO: 16, the LCDR2 of SEQ ID NO: 17 and the LCDR3 of SEQ ID NO: 18; and
(ii) a second antigen binding moiety that binds to EpCAM and comprises a heavy chain variable region comprising the heavy chain CDR (HCDR) 1 of SEQ ID NO: 60, the HCDR2 of SEQ ID NO: 61, and the HCDR3 of SEQ ID NO: 62; and a light chain variable region comprising the light chain CDR (LCDR) 1 of SEQ ID NO: 63, the LCDR2 of SEQ ID NO: 64 and the LCDR3 of SEQ ID NO: 65.

27. The method of claim 26, wherein the T cell bispecific antibody comprises a third antigen binding moiety that binds to EpCAM and/or an Fc domain composed of a first and a second subunit.

28. (canceled)

29. The method of claim 26, wherein the first antigen binding moiety of the T cell bispecific antibody comprises a heavy chain variable region sequence that is at least 98% identical to the amino acid sequence of SEQ ID NO: 14 and a light chain variable region sequence that is at least identical to the amino acid sequence of SEQ ID NO: 19, and/or the second and (where present) third antigen binding moiety of the T cell bispecific antibody comprise a heavy chain variable region sequence that is at least identical to the amino acid sequence of SEQ ID NO: 66 and a light chain variable region sequence that is at least 98% identical to the amino acid sequence of SEQ ID NO: 67.

30. (canceled)

31. (canceled)

32. The method of claim 16, wherein the target cell antigen is tyrosine-related protein 1 (TYRP1); and wherein the cancer is a tyrosine-related protein 1 (TYRP1)-expressing cancer.

33. The method of claim 32, wherein the T cell bispecific antibody comprises:
(i) a first antigen binding moiety that binds to CD3 and comprises a heavy chain variable region comprising the heavy chain CDR (HCDR) 1 of SEQ ID NO: 10, the HCDR2 of SEQ ID NO: 11, and the HCDR3 of SEQ ID NO: 13; and a light chain variable region comprising the light chain CDR (LCDR) 1 of SEQ ID NO: 16, the LCDR2 of SEQ ID NO: 17 and the LCDR3 of SEQ ID NO: 18; and
(ii) a second antigen binding moiety that binds to TYRP1 and comprises a heavy chain variable region comprising the heavy chain CDR (HCDR) 1 of SEQ ID NO: 23, the HCDR2 of SEQ ID NO: 24, and the HCDR3 of SEQ ID NO: 25; and a light chain variable region comprising the light chain CDR (LCDR) 1 of SEQ ID NO: 27, the LCDR2 of SEQ ID NO: 28 and the LCDR3 of SEQ ID NO: 29.

34. (canceled)

35. (canceled)

36. The method of claim 33, wherein the first antigen binding moiety of the T cell bispecific antibody comprises a heavy chain variable region sequence that is at least 98% identical to the amino acid sequence of SEQ ID NO: 15 and a light chain variable region sequence that is at least 98% identical to the amino acid sequence of SEQ ID NO: 19, and/or the second and (where present) third antigen binding moiety of the T cell bispecific antibody comprise a heavy chain variable region sequence that is at least 98% identical to the amino acid sequence of SEQ ID NO: 26 and a light chain variable region sequence that is at least 98% identical to the amino acid sequence of SEQ ID NO: 30.

37. (canceled)

38. (canceled)

39. The method of claim 16, wherein the target cell antigen is a peptide presented on a MHC class I molecule.

40. The method of claim 2, wherein the immunotherapy is a PD-1 axis binding antagonist.

41. The method of claim 40, wherein the PD-1 axis binding antagonist is selected from the group consisting of a PD-1 binding antagonist, a PDL1 binding antagonist and a PDL2 binding antagonist.

42. (canceled)

43. The method of claim 41, wherein the immunotherapy is ipilimumab, nivolumab, pembrolizumab, atezolizumab, durvalumab, or avelumab.

44-46. (canceled)

47. The method of claim 2, wherein the immunotherapy comprises administering chimeric antigen receptor expressing T-cells (CAR T-cells), T-cell receptor (TCR) modified T-cells, tumor-infiltrating lymphocytes (TIL), chimeric antigen receptor (CAR)-modified natural killer cells, T cell receptor (TCR) transduced cells, or dendritic cells, or any combination thereof.

48. The method of claim 2, wherein the immunotherapy comprises administration of a cancer vaccine.

49. The method of claim 18, wherein the cancer is
colorectal cancer, lung cancer, pancreatic cancer, breast cancer, or gastric cancer.

50. The method of claim 25, wherein the cancer is
colorectal, breast, gastric, prostate, ovarian, or lung cancer.

51. The method of claim 32, wherein the cancer is
melanoma.

52. (canceled)
